(19) United States
(12) Patent Application Publication
(10) Pub. No.: US 2002/0119944 A1
(43) Pub. Date: Aug. 29, 2002

(54) Use of Ulip-and/or Ulip2 in the treatment of myelin disorders

(76) Inventors: Michelle Aguera, Caluire (FR); Marie-Francoise Belin, Lyon (FR); Emmanuelle Charrier, Les Herbiers (FR); Jerome Honorat, Brion (FR); Damien Ricard, Lyon (FR); Veronique Rogemond, Lyon (FR)

Correspondence Address: LARSON & TAYLOR, PLC, 1199 NORTH FAIRFAX STREET, SUITE 900, ALEXANDRIA, VA, 22314, US

(21) Appl. No.: 09/986,632

(22) Filed: Nov. 9, 2001

Related U.S. Application Data
(60) Provisional application No. 60/246,751, filed on Nov. 9, 2000.

(51) Int. Cl. A61K 48/00; A61K 38/17; A61K 39/395

(52) U.S. Cl. 514/44; 514/12; 424/146.1

(57) ABSTRACT

This invention relates to methods and compositions for the prevention or treatment of myelin disorders which involve modulating of Ulip/CRMP activity. In particular, a method for the prevention or treatment of myelin disorders is provided which comprises administering to a patient in need of such treatment a therapeutically effective amount of an agent which modulates Ulip/CRMP activity, and which can be a Ulip/CRMP protein, a nucleic acid coding for a Ulip/CRMP protein, an anti-sense sequence capable of specifically hybridizing with said nucleic acid, an antibody directed against the Ulip/CRMP protein, or an aptamer capable of binding said protein, and a pharmacologically acceptable carrier.

### CROSS-REFERENCE TO RELATED APPLICATIONS

This application claims the benefit of U.S. provisional application No. 60/246,751, filed Nov. 9, 2000.

### FIELD OF THE INVENTION

The present invention relates to the modulation of Ulip/CRMP activity in the prevention or treatment of myelin disorders.

### BACKGROUND OF THE INVENTION

A new protein family with four members, homologous to the UNC-33 protein required in nematodes for appropriate axonal and synaptic organization (Li et al., 1992), has recently been identified in human, rat, mouse, and chicken brain. These proteins, known variously as TOAD-64 (Turned On After Division) (Minturn et al., 1995a), CRMP (Collapsin Response Mediator Protein) (Wang and Strittmatter, 1996), DRP (Dihydropyrimidinase Related Protein) (Hamajima et al., 1996), C-22 (Quach et al., 1997), or Ulip (Unc-33-Like Protein) (Byk et al., 1998, WO 98137 192), are highly expressed by neural cells in the developing brain, the highest expression being seen, in the rodent, at the end of embryonic life during the period of maximal axonal growth (Minturn et al., 1995a; Wang and Strittmatter, 1996). Members of this protein family are presumed to be involved in axonal outgrowth in response to Sema3A (Goshima et al., 1995; Semaphorin Nomenclature Committee, 1999), a member of the semaphorin protein family (Kolodkin et al., 1997).

Although dramatically downregulated in the adult, Ulip/CRMPs are still expressed in structures that retain neurogenesis (Wang and Strittmatter, 1996; Kamata et al., 1998 ; Pasterkamp et al., 1998; Nacher et al., 2000). Interestingly, members of the Ulip/CRMP family have been implicated in human neurodegenerative disorders. In Alzheimer's disease, increased levels of highly phosphorylated Ulip2/CRMP2 are associated with neurofibrillary tangles (Yoshida et al., 1998; Gu et al., 2000). In Paraneoplastic Neurological Diseases (PND), autoimmune neurodegenerative disorders involving the cerebellum and dentate gyrus, some patients develop autoantibodies (anti-CV2 antibodies) recognizing Ulip/CRMP proteins (Honnorat et al., 1999). Intriguingly, although all anti-CV2 sera tested recognized the same protein (Honnorat et al., 1996) and immunolabeled the same postmitotic neural precursors in the developing brain and the same population of adult oligodendrocytes (Honnorat et al., 1998), a few failed to recognize any of the four known Ulip/CRMPs, suggesting the existence of another member that was the main target for these antibodies, and that was referred to as Ulip6/CRMP5. This protein displays 50% homology with the other human Ulip/CRMPs and is the human equivalent of the CRAM and CRMP5 proteins, recently identified, respectively, in the rat and mouse (Fukada et al., 2000; Inatome et al., 2000).

### SUMMARY OF THE INVENTION

The authors of the present invention have now shown that the Ulip/CRMP protein family, and more particularly the newly identified Ulip6/CRMP5 and/or Ulip2/CRMP2, is involved in myelination, demyelination and remyelination in central nervous system.

The present invention thus provides a method for the prevention or treatment of myelin disorders, comprising modulating Ulip/CRMP activity.

The invention more particularly provides a method for the prevention or treatment of myelin disorders, comprising administering to a patient in need of such treatment a therapeutically efficient amount of an agent selected from the group consisting of a purified Ulip protein, preferably a Ulip6/CRMP5 and/or Ulip2/CRMP2 protein, a nucleic acid encoding said protein, an anti-sense sequence capable of specifically hybridizing with said nucleic acid, and an antibody directed against said protein, in association with a pharmaceutically suitable carrier.

The present invention also provides a method of diagnosis of a myelin disorder, wherein the expression of a Ulip/CRMP protein, in particular Ulip6/CRMP5 and/or Ulip2/CRMP2 or the presence of antibodies anti-Ulip/CRMP, in particular anti-Ulip6/CRMP5 and/or anti-Ulip2/CRMP2, is evaluated in a biological sample of a patient to be tested and is compared to the levels present in normal subjects.

Methods of screening agents that modulate Ulip/CRMP activity are further encompassed.

### BRIEF DESCRIPTION OF THE DRAWINGS

FIG. 1: Western-blot revealed with anti-Ulip6/CRMP5, anti-Ulip2/CRMP2 and anti-CV2 sera. A: western blot showing specific binding of anti-Ulip6/CRMP5, anti-Ulip2/CRMP2, or anti-Ulip3/CRMP1 antibodies to the corresponding Ulip/CRMP recombinant protein. Rat brain extract (rat brain) was used as a positive control for Ulip2/CRMP2, Ulip6/CRMP5 and Ulip3/CRMP1 expression in brain. B: western blot with Ulip6/CRMP5 recombinant protein showing that anti-CV2 sera from 12 PND patients recognized this protein (lanes 1-12). Lane 13 shows the lack of binding of a representative control serum. U1/C4: Ulip1/CRMP4; U2/C2: Ulip2/CRMP2; U3/C1: Ulip3/CRMP1; U4/C3: Ulip4/CRMP3; U6/C5: Ulip6/CRMP5.

FIG. 2: Ulip6/CRMP5 expression using Northern and Western blots. A: northern blot of adult human tissue showing specific expression of Ulip6/CRMP5 mRNA in the brain (5.5 kb) (pbl: peripheral blood leukocytes). B: western blot of cerebellum extracts from E19, P5, and adult (Ad) rats showing Ulip6/CRMP5 protein expression is maximal at El 9, lower at P5, and weak in the adult. C: western blot of one-day-old rat tissue extracts showing Ulip6/CRMP5 protein is expressed at a high level in brain and at a lower level in muscle. D: western blot of adult rat tissue extracts showing Ulip6/CRMP5 protein is expressed in brain and testis.

FIG. 3: Expression of Ulip6/CRMP5 mRNA and protein in the embryonic rat brain. Sagittal sections (14 pm) of E19 dorsal telencephalon (A, B) or frontal sections (14 pm) of E16 spinal cord (C, D) were hybridized with the Ulip6/CRMP5 ripobrobe (A, C) or immunolabeled with anti-Ulip6/CRMP5 antibodies (B, D). Expression of Ulip6/CRMP5 mRNA (A, C) or protein (B, D) was never detected in the neuroepithelium zone of the cerebral neocortex and spinal cord (star). Ulip6/CRMP5 mRNA and protein were highly expressed in the differentiating field of the neocortex (nc), hippocampus (hc) (A, B), spinal cord (sc), and dorsal root ganglia (drg) (C, D). Ulip6/CRMP5 protein was especially strongly expressed in the hippocampal fimbria (arrowhead) (B), spinal tracts, and peripheral nerves (arrows) (D). No Ulip6/CRMP5 mRNA or protein was detected in the basal ganglia (bg) (A, B). Scale bar =330 pm.

FIG. 4: Expression of Ulip6/CRMP5 and Ulip2/CRMP2 mRNAs in the developing rat cerebellum. Sagittal sections (14 μm) of E19 (A, D), P15 (B, E), and adult (C, F) rat cerebellum were hybridized with the Ulip6/CRMP5 (A, B, and C) or Ulip2/CRMP2 (D, E, and F) riboprobes. At E19, Ulip6/CRMP5 (A) and Ulip2/CRMP2 (D) mRNAs were detected in the migrating cells under the EGL (white arrows) and in the deep nuclei (white arrowhead). Only Ulip2/CRMP2 mRNA (D) was expressed in the EGL (egl). At P15, both Ulip6/CRMP5 (B) and Ulip2/CRMP2 (E) mRNAs were expressed in the internal part of the EGL (white arrowhead). Expression of Ulip6/CRMP5 mRNA and, to a lesser extent, Ulip2/CRMP2 mRNA was seen in the molecular layer (ml) and IGL (igl). Only Ulip2/CRMP2 mRNA was detected in the external part of the EGL (thin black arrow), the Purkinje cells layer (pl), and oligodendrocytes of the white matter (thick black arrow). In the adult cerebellum, expression of Ulip6/CRMP5 mRNA (C) was detected in the Purkinje cells layer (pl), oligodendrocytes of the white matter (wm, black arrow), and, to a lesser extent, in the molecular layer (ml) and internal granular layer (igl). Ulip2/CRMP2 mRNA (F) was still expressed in the Purkinje cell layer (pl), oligodendrocytes of the white matter (wm, black arrow), and, to a lesser extent, in the molecular layer (ml) and internal granular layer (igl). A, D, C, and F: Scale bar=120 μm. B, E: Scale bar=90 μm.

FIG. 6: Expression of Ulip6/CRMP5 and Ulip2/CRMP2 proteins in the developing rat cerebellum. Sagittal sections (14 μm) of E19 (A, B), P15 (C, D), or adult (E, F) rat cerebellum were immunolabeled with anti-Ulip6/CRMP5 (A, C, and E) or anti-Ulip2/CRMP2 (B, D, and F) antibodies. At E19, Ulip6/CRMP5 protein (A) was expressed in all layers of the cerebellum except the EGL (egl), while Ulip2/CRMP2 protein (B) was detected in the EGL (egl) and, to a lesser extent, in the region under the EGL (arrows). At P15, only Ulip2/CRMP2 protein (D) was detected in the external part of the EGL (thin arrow) and in the Purkinje cell layer (pl), while both Ulip6/CRMP5 (C) and Ulip2/CRMP2 (D) proteins were expressed in the internal part of the EGL (arrowhead) and in the molecular layer (ml). Double-labeling showed coexpression of Ulip6/CRMP5 (C) and Ulip2/CRMP2 (D) in neural precursors of the internal EGL (insert, arrow). Ulip6/CRMP5 protein (C) and, to a lesser extent, Ulip2/CRMP2 protein (D) were detected in the IGL (igl) and the white matter (thick arrow). In the adult cerebellum, expression of Ulip6/CRMP5 (E) and Ulip2/CRMP2 (F) proteins was only detected in the oligodendrocytes of the white matter (wm, arrow). A, B: Scale bar=180 μm. C, D: Scale bar =90 pm. C, D; insert: Scale bar 15 μm. E, F: Scale bar=40 μm.

FIG. 6: Expression of Ulip6/CRMP5 mRNA and protein in adult rat brain. Sagittal sections (14 μm) of the frontal cortex (A, B), hippocampus (C, D), or spinal cord (E, F) were hybridized with the Ulip6/CRMP5 riboprobe (A, C, and E) or immunolabeled with anti-Ulip6/CRMP5 antibodies (B, D, and F). Both mRNA (A, C) and protein (B, D) were expressed in neurons of the frontal cortex (A, B) and hippocampus (C, D), especially in the infragranular layer (arrow). Both mRNA (E) and protein (F) were also expressed in oligodendrocytes of the spinal cord (arrowhead). A: Scale bar=60 μm. B: Scale bar=30 pm. C: Scale bar=310 μm. D: Scale bar=50 pm. E: Scale bar=40 μm. F: Scale bar=25 μm.

FIG. 7: Expression of Ulip6/CRMP6 and Ulip2/CRMP2 mRNAs and proteins in oligodendrocytes of the adult rat spinal cord. Sagittal sections (14 μm) of the adult rat spinal cord were immunolabeled with both rabbit anti-Ulip6/CRMP5 antibodies (A) and rat anti-Ulip2/CRMP2 antibodies (B). All oligodendrocytes labeled by anti-Ulip6/CRMP5 antibodies expressed Ulip2/CRMP2 protein (arrow). A few oligodendrocytes expressing Ulip2/CRMP2 protein were negative for Ulip6/CRMP5 protein (arrowhead). Frontal sections (14 μm) of adult rat spinal cord were hybridized with the Ulip6/CRMP5 (C) or Ulip2/CRMP2 (D) riboprobes. Oligodendrocytes of the internal part of cortico-spinal tract expressing Ulip2/CRMP2 mRNA were negative for Ulip6/CRMP5 mRNA (arrows). A, B: Scale bar=30 μm. C, D: Scale bar=200 pm.

FIG. 8: Sema3A binding and neuropilin-1 mRNA expression in purified adult rat brain oligodendrocytes. A, B: AP-Sema3A binding sites visualized on oligodendrocytes using AP staining solution (A) and labeling with Rip antibody (B). C, D: AP-Sema3A binding was blocked by an excess of Sema3A on purified oligodendrocytes (C) immunolabeled with Rip antibody (D). E, F: expression of neuropilin-1 mRNA on oligodendrocytes by in situ hybridization with anti-sense probe (E) and labeled with Rip antibody (F). G, H: in situ hybridization using the neuropilin-1 sense probe showed absence of signal (G) on oligodendrocytes immunolabeled with Rip antibody (H). Scale bar=24 μm.

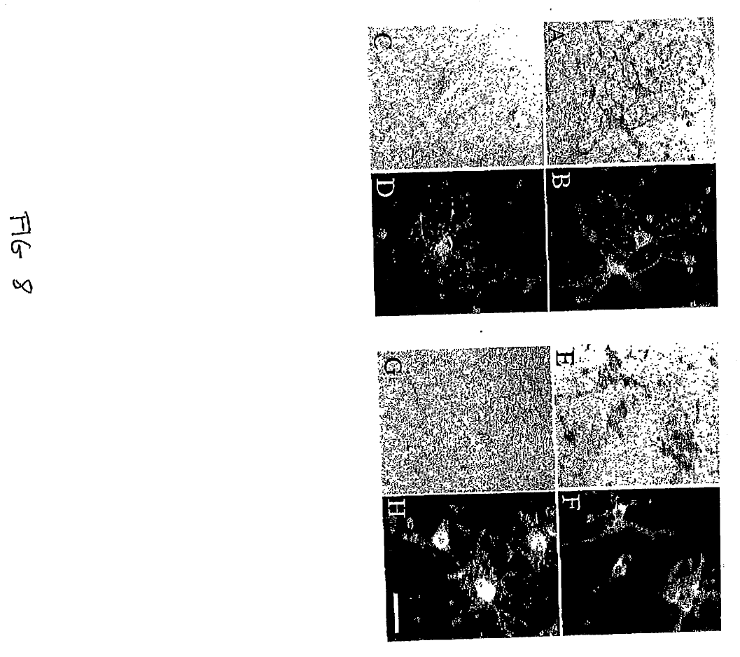

FIG. 9: Sema3A inhibition of process extension by Ulip6/CRMP5-expressing adult rat brain oligodendrocytes. A, B: immunolabeling of Ulip6/CRMP5 protein on oligodendrocytes (A) double labeled with Rip antibody (B). C: purified oligodendrocytes grown 24 h in control medium, showing process extension, immunolabeled with Rip antibody. D: oligodendrocytes cultured in a Sema3A-conditioned medium, showing an absence of process extension, immunolabeled with Rip antibody. E: oligodendrocytes immunolabeled with Rip antibody treated with Sema3A medium as in D, followed by removal of the Sema3A medium and incubation for 48 h in control medium showing restoration of process extension. A, B: scale bar=40 μm. C, D, E: scale bar=30 μm.

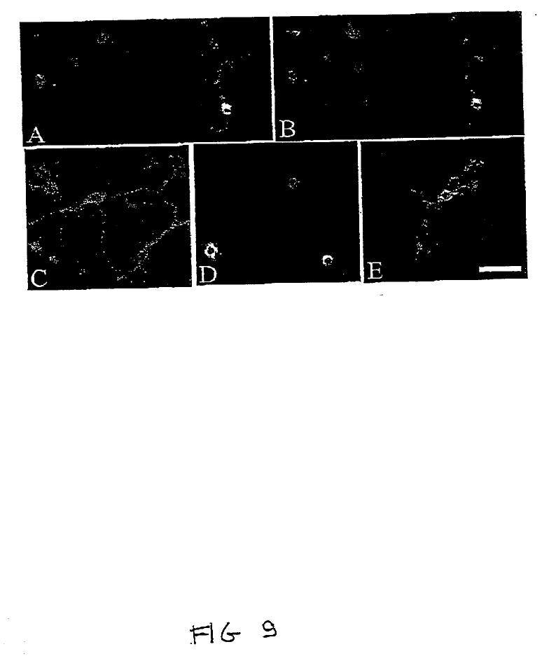

FIG. 10: Quantitative evaluation of oligodendrocyte process extension. Concentric circles separated by 10 pm were drawn around the cell bodies of the microphotographed oligodendrocytes. Intersections of the oligodendrocyte processes with the concentric circles were counted to define a branching index (BI).

FIG. 11: Quantitative effect of Sema3A on purified adult rat brain oligodendrocytes. A: time-course of the Sema3A effect on the oligodendrocyte branching index. The cells were incubated for 24, 48, and 72 h with Sema3A-conditioned medium (Sema3A) or control medium (control) and the branching index compared (*=p <0.0001). B: dose-response curve for the effect of Sema3A on the branching index. Oligodendrocytes were cultured for 48 h in control medium (0) or different dilutions of Sema3A-conditioned medium in control medium (100% and 1% represent, respectively, undiluted and a 1/100 dilution of Sema3A-conditioned medium). C: Effect of VEGF-165 or anti-neuropilin-1 antibodies on the branching index of purified oligodendrocytes cultured in the presence of Sema3A. Cells were incubated with a ⅕ dilution of Sema3A-conditioned medium in control medium in the presence of VEGF-165 (+VEGF) or anti-neuropilin-1 antibodies (+anti-neurop) and with control medium (control) (*=p<0.001). D: Effect of anti-Ulip2/CRMP2 and anti-Ulip6/CRMP5 antibodies on the branching index of purified oligodendrocytes cultured in the presence of Sema3A. Purified oligodendrocytes were cultured in Sema3A-conditioned medium in the presence of anti-Ulip2/CRMP2 (anti-U2/C2, 4, 8 or 20 μg/ml), anti-Ulip6/CRMP5 (anti-U6/C5, 2, 4 or 8 μg/ml), or anti-Ulip3/CRMP1 (anti-U3/C1, 8 μg/ml) antibodies or preimmune IgG (8 pg/ml) to block the Sema3A effect. The data are the mean±SD (bars) values for 20 cells in each case. The branching index for each condition was compared to the branching index obtained in the presence of Sema3A alone (*: p<0.001).

### DETAILED DESCRIPTION

To investigate the putative function of Ulip/CRMP proteins in oligodendrocyte cells, the authors of the present invention have analyzed the pattern of expression of the five Ulip/CRMP (Ulip3/CRMP1, Ulip2/CRMP2, Ulip4/CRMP3, Ulip1/CRMP4, and Ulip6/CRMP5) transcripts in the adult rodent CNS. They have shown by in situ hybridization that Ulip2/CRMP2 mRNA is highly expressed in mature myelin-forming oligodendrocytes. Using an anti-Ulip2/CRMP2 antiserum, they also confirmed that, in vivo, the protein is present in oligodendrocytes, but not in astrocytes. Transcripts encoding the other Ulip/CRMP members are also detected by RT-PCR in highly purified mature oligodendrocytes.

They further compared the distribution of Ulip2/CRMP2 and Ulip6/CRMP5 and found that they were coexpressed at certain times during development and in oligodendrocytes. In studies to understand the function of Ulip6/CRMP5 and Ulip2/CRMP2 in adult, purified adult rat brain oligodendrocytes were submitted to Sema3A, a semaphorin mainly known for its attractive/repulsive properties on growing axons (Bagnard et al., 1998, 2000). These oligodendrocytes were found to have Sema3A binding sites and to express neuropilin-1, the major component of the Sema3A receptor complex (He and Tessier-Lavigne, 1997; Kolodkin et al., 1997). In the presence of Sema3A, the oligodendrocyte process extensions displayed a dramatic decrease which was reversed by removing the Sema3A or prevented by anti-neuropilin-1, anti-Ulip6/CRMP5, anti-Ulip2/CRMP2 antibodies or VEGF-165, another ligand for neuropilin-1 (Miao et al., 1999). These results indicate the existence of a Sema3A signaling pathway controlling oligodendrocyte process extension in adult brain via Ulip6/CRMP5 and/or Ulip2/CRMP2, and support the involvement of Ulip6/CRMP5 and/or Ulip2/CRMP2 in myelination, demyelination and remyelination in the normal and pathological central nervous system. Ulip6/CRMP5 and/or Ulip2/CRMP2 are more particularly involved in myelination or remyelination after injury when oligodendrocytes must develop processes and choose their axonal targets. The observation of other members of Ulip/CRMP family in oligodendrocytes further supports the Ulip/CRMP in myelin disorders.

The definitions given hereafter equally apply to all sections of the described invention.

Myelin disorders:

“Myelin disorders” include, but are not limited to, multiple sclerosis, HTLV1-associated myelopathy, and leucodystrophies.

In multiple sclerosis, as well as in other demyelinating disorders, before oligodendrocytes can remyelinate, they must extend and contact the demyelinated axons. The role of Ulip/CRMP, in particular Ulip2/CRMP2 and Ulip6/CRMP5 in the response to signals, such as Sema3A, could be crucial in the reinitiation and regulation of process extension by surviving oligodendrocytes. In addition, when axons are injured, levels of Sema3A (Pasterkamp et al., 1998) and Ulip2/CRMP2 (Minturn et al., 1995; Pasterkamp et al., 1998) are altered. Thus, both oligodendrocyte process extension and axonal regrowth could be dramatically altered in response to Sema3A when the brain is injured.

ULIP:

The Ulip proteins family, also known as CRMP proteins, now comprises five different members.

According to the present invention, the “Ulip6/CRMP5 protein” refers to a protein substantially comprising the amino-acid sequence shown in SEQ ID n° 2, as well as polypeptide fragments and derivatives thereof. A nucleic acid sequence coding for the Ulip6/CRMP5 protein comprises the nucleic acid sequence from nucleotides 163 to 1854 in SEQ ID n° 1, or degenerates thereof.

The “Ulip2/CRMP2 protein” refers to a protein substantially comprising the amino-acid sequence shown in SEQ ID n° 4 (also available on EMBL/Genbank database under access number U 17279), as well as polypeptide fragments or derivatives thereof. A nucleic acid sequence coding for the Ulip2/CRMP2 protein comprises the nucleic acid sequence from nucleotides 72 to 1790 in SEQ ID n° 3, or degenerates thereof.

The “Ulip1/CRMP4 protein” refers to a protein substantially comprising the amino-acid sequence shown in SEQ ID n° 6 (also available on EMBL/Genbank database under access number Y 07818), as well as polypeptide fragments or derivatives thereof. A nucleic acid sequence coding for the Ulip/CRMP4 protein comprises the nucleic acid sequence SEQ ID n° 5, or degenerates thereof.

The “Ulip3/CRMP1 protein” refers to a protein substantially comprising the amino-acid sequence shown in SEQ ID n° 8 (also available on EMBL/Genbank database under access number D 78012), as well as polypeptide fragments or derivatives thereof. A nucleic acid sequence coding for the Ulip3/CRMP1 protein comprises the nucleic acid sequence SEQ ID n° 7, or degenerates thereof.

The “Ulip4/CRMP3 protein” refers to a protein substantially comprising the amino-acid sequence shown in SEQ ID n° 10, as well as polypeptide fragments or derivatives thereof. A nucleic acid sequence coding for the Ulip4/CRMP3 protein comprises the nucleic acid sequence SEQ ID n° 9, or degenerates thereof.

In a preferred embodiment, both Ulip6/CRMP5 and Ulip2/CRMP2 are targeted in the treatment of myelin disorders.

Derivative polypeptide refers to any variant polypeptide of the proteins above or any other molecule resulting from a modification of genetic and/or chemical nature of the sequence SEQ ID n° 2, n° 4, no 6, n° 8, or n° 10, that is to say obtained by mutation, deletion, addition, substitution and/or chemical modification of a single or of a limited number of amino acids, as well as any isoform sequence, the said modified or isoform variant sequences having conserved at least one of the properties making them biologically active.

The invention likewise relates to the use of an isolated nucleic acid sequence selected from SEQ ID n° 1, n° 3, n° 5, n° 7, or n° 9, or a nucleotide fragment or derivative sequences derived from the sequences SEQ ID n° 1, n° 3, n° 5, n° 7, or n° 9, on account of the degeneracy of the genetic code, or on account of mutation, of deletion or of insertion of at least one nucleotide.

The various nucleotide sequences of the invention can be of artificial or non-artificial origin. They can be DNA or RNA sequences.

The derivative nucleotide sequences also include sequences capable of hybridizing strongly and specifically with SEQ ID n° 1, n° 3, n° 5, n° 7, or n° 9 or their complementary sequences. The appropriate hybridization conditions correspond to the conditions of temperature and of ionic strength usually used by the person skilled in the art (Sambrook et al, 1989), preferably to temperature conditions of between Tm minus 5C and Tm minus 30° C and more preferably to temperature conditions of between Tm minus 5° C and Tm minus 10° C. (great stringency), Tₘ being the theoretical melting point, defined as being the temperature at which 50% of the paired strands separate.

The nucleotide sequences SEQ ID n° 1, n° 3, n° 5, n° 7, or n° 9 are useful for the production of antisense sequences capable of hybridizing specifically with a nucleic acid sequence, including a messenger RNA, which can be used in gene therapy.

Therapeutic Methods:

The present invention provides a method for the prevention or treatment of myelin disorders, comprising modulating a Ulip/CRMP activity. Preferably said Ulip/CRMP is Ulip6/CRMP5 and/or Ulip2/CRMP2.

In the context of the present invention, “prevention” of myelin disorder may be more particularly aimed at patients that have not shown any symptoms of the disease but that may be susceptible or predisposed to develop the disease.

“Treatment” means therapeutic treatment of patients to alleviate myelin disorder at any stage of development of the disease.

In a preferred embodiment, the patient is human, preferably an adult, but the methods according to the present invention can also be applied to mammals or other vertebrates.

“Modulating a Ulip/CRMP activity” is intended for enhancing or inhibiting the activity of said Ulip/CRMP protein in a myelin disorder condition.

The “activity” of Ulip/CRMP proteins includes any biological property of the protein. Or instance, such activity may be assessed by evaluating the axonal or oligodendrocyte outgrowth inhibition in response to Semaphorins, in particular Sema3A. It also includes immunological properties of the Ulip/CRMP proteins and it refers particularly to eliciting anti-CV2 antibodies in Paraneoplastic Neurological Diseases.

Such a modulation of Ulip/CRMP activity can be direct or indirect.

A “direct” modulation of a Ulip/CRMP protein activity, is a modulation that is carried out through directly acting on the activity and/or expression of the Ulip/CRMP protein itself.

Agents capable of directly modulating the Ulip/CRMP protein activity are either agonist or antagonists and can also be designated as “direct activators” or “direct inhibitors”, respectively. An agonist is thus intended for an agent that enhances the activity whereas an antagonist is intended for an agent that inhibits the activity of a protein. In a particular embodiment, such agonists or antagonists are capable of modulating the interaction of the Ulip/CRMP protein with endogenous molecules that usually act directly upstream or downstream the Ulip/CRMP protein within a signalization cascade. Such agents are for instance antibodies directed against said Ulip/CRMP protein or aptamers.

Altering interaction between two homologous or heterologous Ulip/CRMP proteins is another example of modulation of Ulip/CRMP activity.

Interaction between “homologous Ulip/CRMP” proteins is intended for interaction between at least two same types of Ulip/CRMP proteins, such as homodimers Ulip2/CRMP2-Ulip2/CRMP2.

Interaction between “heterologous Ulip/CRMP” proteins is intended for interaction between at least two different Ulip/CRMP proteins, such as heterodimers Ulip2/CRMP2-Ulip6/CRMP5.

Among agents capable of directly modulating the Ulip/CRMP expression, one can cite agents that alter (i.e. enhance or diminish) the level of production of the Ulip/CRMP protein. Such agents can be for example a Ulip/CRMP polypeptide or a nucleic acid sequence coding for said protein, or agents capable of modulating the transcription and/or translation of Ulip/CRMP genes, such as anti-sense nucleic acid sequences.

A “indirect” modulation of a Ulip/CRMP protein activity, is a modulation that is carried out through acting on the expression or activity of any extracellular or intracellular endogenous agents that usually act upstream (“inducer”) or downstream (“effector”) the Ulip/CRMP protein within a signalization cascade. Accordingly an inducer of a Ulip/CRMP protein is for instance a Semaphorin, in particular Semaphorin 3A (Sema3A) or Semaphorin 4D (Sema4D). Examples of effectors include tyrosine kinases, Rho family GTPase or Rac. Other proteins capable of interacting with Ulip/CRMP proteins which can be identified in pathological samples, such as cerebro-spinal fluid or brain tissues, from a patient (human or animal) affected with a myelin disorder, are within the scope of the present invention. Agents allowing to achieve indirect modulation of activity or expression of a Ulip/CRMP can be readily selected by one skilled in the art, for instance in view of the above described types of direct modulators.

In the context of the present invention, a “Ulip/CRMP signalization cascade” refers in particular the Sema3A induced axonal or oligodendrocyte outgrowth inhibition.

According to the present invention, and unless otherwise specifically defined, the term “agents” or “test compounds” can refer to one or more structurally defined molecules such as polypeptides, oligonucleotides, organic or mineral molecules, of endogenous or exogenous nature. Agents can also be undefined compounds such as cellular, tissue or biological liquid extracts from animal or vegetal origin.

In particular, the present invention relates to a method for the prevention or treatment of myelin disorders, comprising administering to a patient in need of such treatment a therapeutically effective amount of an agent selected from the group consisting of a Ulip/CRMP protein, a nucleic acid coding for a Ulip/CRMP protein, an anti-sense sequence capable of specifically hybridizing with said nucleic acid, an antibody directed against the Ulip/CRMP protein, and an aptamer capable of binding said protein, and a pharmacologically acceptable carrier.

In a preferred embodiment, said Ulip/CRMP protein is Ulip6/CRMP5 and/or Ulip2/CRMP2. Preferably, the nucleic acid according to the invention may be a nucleic acid coding for the Ulip6/CRMP5 protein that comprises the nucleic acid sequence from nucleotides 163 to 1854 in SEQ ID n° 1, or degenerates thereof. Also preferably, the nucleic acid according to the invention may be a nucleic acid coding for the Ulip2/CRMP2 protein that comprises the nucleic acid sequence from nucleotides 72 to 1790 in SEQ ID n° 3, or degenerates thereof.

Preferably, said active agent is purified.

Another subject of the present invention is the use of an agent as above-described for the manufacture of a pharmaceutical composition suitable for the prevention or treatment of myelin disorders.

In a preferred embodiment, blocking Ulip/CRMP, and in particular Ulip6/CRMP5 and/or Ulip2/CRMP2 is searched. In that case, the method of the present invention may use antisense sequences or antibodies anti-Ulip6/CRMP5 and/or anti-Ulip2/CRMP2.

One may also use a compound or a mixture of compounds of synthetic or natural origin that inhibits the action of said Ulip/CRMP proteins, more particularly by blocking the interaction between two of them. In a preferred embodiment, said Ulip/CRMP protein is Ulip6/CRMP5 and/or Ulip2/CRMP2.

Alternatively, enhancing the expression and/or activating of a Ulip/CRMP protein, in particular Ulip6/CRMP5 and/or Ulip2/CRMP2 may be searched. In that case, the method of the present invention may use a nucleic acid encoding said proteins or a purified isolated Ulip/CRMP protein. One may also use a compound or a mixture of compounds of synthetic or natural origin that activates or enhances the expression or action of a Ulip/CRMP protein. Compounds that stimulate the interaction between Ulip/CRMP proteins, especially Ulip6/CRMP5 and Ulip2/CRMP2, are therefore preferred.

Such stimulatory or inhibitory compounds may be selected by a screening method wherein a compound to be tested is contacted with Ulip/CRMP proteins and the interaction between two proteins is determined. Preferably, said screening involves contacting with Ulip6/CRMP5 and/or Ulip2/CRMP2.

Screening methods are described in greater details hereafter.

The invention thus provides antisense nucleic acids (including ribozymes), which may be used to inhibit expression of a target protein of the invention. An “antisense nucleic acid” is a single stranded nucleic acid molecule, which, on hybridizing under cytoplasmic conditions with complementary bases in a RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA6RNA interactions, RNA-DNA interactions, ribozymes and Rnase-H mediated arrest. Antisense nucleic acid molecules can be encoded by a recombinant gene for expression in a cell (e.g., U.S. Pat. Nos. 5,814,500; 5,811,234), or alternatively they can be prepared synthetically (e.g., U.S. Pat. No. 5,780,607).

The invention likewise relates to mono- or polyclonal antibodies directed against Ulip/CRMP polypeptide comprising an amino acid sequence selected from SEQ ID n° 2, n° 4, n° 6, n° 8, or n° 10.

Polyclonal antibodies can be obtained from the serum of an animal immunized against the protein, produced, for example, by genetic recombination according to the usual working methods.

The monoclonal antibodies can be obtained according to the conventional method of hybridoma culture described by Köhler and Milstein.

The antibodies can be chimeric antibodies, humanized antibodies, Fab and F(ab′)₂ fragments. They can likewise be present in the form of immunoconjugates or labeled antibodies.

Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999.

Thus, the invention also relates to aptamers directed against Ulip/CRMP polypeptide comprising an amino acid sequence selected from SEQ ID n° 2, n° 4, n° 6, n° 8, or n° 10.

The compounds that may be useful for treating or preventing a myelin disorder can be determined with the help of a system for which comprises:

a testing means which allows one to contact a test compound suspected of having a stimulatory or inhibitory activity on a Ulip/CRMP protein with a Ulip/CRMP protein; and

a determining means to determine if the test compound has a stimulatory or inhibitory activity on the Ulip/CRMP protein, said activity being indicative of a compound potentially useful for treating or preventing a myelin disorder.

In such a system the Ulip/CRMP protein is preferably a Ulip2/CRMP2 protein and/or a Ulip6/CRMP5 protein.

Diagnostic Methods:

The diagnostic methods described hereafter can be useful for detecting a myelin disorder in human or animal subjects. They may be performed for asymptomatic subjects or subjects with a suspicion of myelin disorder. Subjects who are predisposed to developing a myelin disorder naturally are a preferred target.

The present invention also provides a method of prognosis and/or diagnosis of a myelin disorder in a subject, comprising:

evaluating the level of expression of at least one agent selected from the group consisting of a Ulip/CRMP protein and antibodies to a Ulip/CRMP protein present in the sample in a biological sample from said subject;

comparing the level of expression of said agent in the biological sample with expression levels of said agent in control subjects.

In a preferred embodiment, the level of expression of Ulip2/CRMP2 and/or Ulip6/CRMP5 protein or antibodies thereto is evaluated.

Alternatively, the presence of antibodies anti-Ulip/CRMP may be determined by means of Ulip/CRMP proteins or epitopic fragments thereof, that can be detectably labelled so that the immune complexes formed between said proteins and said antibodies are easily detected in a biological sample.

Methods for producing antibodies as described in the “Therapeutic methods” section can also be easily adapted to produce antibodies useful for the diagnostic methods according to the invention.

The biological sample wherein a Ulip/CRMP protein or an anti-Ulip/CRMP antibody could be detected is for instance a biological liquid, such as blood or spinal fluid, or a tissue biopsy.

Screening Methods:

In another aspect, the present invention relates to a method for identifying agents useful for the prevention or treatment of myelin disorders, comprising

contacting a Ulip/CRMP protein or a Ulip/CRMP expressing cell with a test compound;

determining if the test compound has a modulatory effect on the Ulip/CRMP activity; and

identifying those test compounds having a stimulatory or inhibitory effect on the Ulip/CRMP protein, as useful for the prevention or treatment of myelin disorders.

According to a particular embodiment, the modulatory effect of the test compound is assessed by evaluating the level of expression of the Ulip/CRMP protein. The methods allowing to assessed the level of expression of a protein are readily known by one skilled in the art.

In the context of the present application, the Ulip/CRMP expressing cell is a cell that displays endogenous expression of the Ulip/CRMP protein or a host cell that has been transformed to express said protein.

Preferably said cell is an oligodendrocyte. In this case, the modulatory effect of the test compound can be assessed for instance by an oligodendrocyte process extension assay such as calculating the branching index, as herein described in the following examples.

Still preferably, the above method relies on the identification of Ulip2/CRMP2 and/or Ulip6/CRMP5 activity modulating agents.

Accordingly, the present invention also relates to a Ulip/CRMP activity modulatory agent useful for the prevention or treatment of myelin disorders as can be identified by the above described method.

The present invention also provides a method for identifying agents, useful for the prevention or treatment of myelin disorders comprising:

contacting a Ulip/CRMP protein and an inducer or effector protein with a test compound in a suitable medium allowing the interaction between the Ulip/CRMP protein and its inducer or effector protein;

determining if the test compound has a stimulatory or inhibitory effect on the interaction between the Ulip protein and its inducer or effector protein; and

identifying those test compounds having a stimulatory or inhibitory effect on the interaction between the Ulip/CRMP protein and its inducer or effector protein, as useful for the prevention or treatment of myelin disorders.

In a preferred embodiment, the Ulip/CRMP protein is a Ulip2/CRMP2 protein or a Ulip6/CRMP5 protein.

Pharmaceutical Compositions:

The agents identified by the above methods also belong to the invention. In particular such an agent can be useful to prepare a composition, for treating or preventing a myelin disorder, comprising administering said agent in association with a pharmaceutically acceptable carrier.

Therefore, the invention also relates to a method of treating or preventing a myelin disorder comprising administering to a patient in need of such treatment a therapeutically effective amount of a composition according to the invention.

Other useful pharmaceutical compositions comprise an agent selected from the group consisting of a Ulip/CRMP protein, a nucleic acid coding for a Ulip/CRMP protein, an anti-sense sequence capable of specifically hybridizing with said nucleic acid, an antibody directed against the Ulip/CRMP protein, and an aptamer capable of binding said protein, and a pharmacologically acceptable carrier.

Pharmaceutical compositions of the invention, may be administered to a mammal, preferably to a human being, in need of a such treatment, according to a dosage which may vary widely as a function of the age, weight and state of health of the patient, the nature and severity of the complaint and the route of administration. The appropriate unit forms of administration comprise oral forms such as tablets, gelatin capsules, powders, granules and oral suspensions or solutions, sublingual and buccal administration forms, cutaneous, subcutaneous, intramuscular, intravenous, intranasal or intraocular administration forms and rectal administration forms.

Pharmaceutical compositions may contain suitable pharmaceutically acceptable carriers comprising excipients and auxiliaries which facilitate processing of the active compounds into preparations which can be used pharmaceutically. Preferred compositions include the inclusion of an adjuvant, such as alum, or other adjuvants known in the art.

To enhance delivery or bioactivity, the polypeptides can be incorporated into liposomes using methods and compounds known in the art.

According to a specific embodiment of the invention, polynucleotides are administered into a patient to achieve controlled expression of the Ulip/CRMP protein.

Said polynucleotides are DNA or RNA sequences encoding the Ulip/CRMP protein, operatively linked to the genetic elements necessary for their expression by a target cell, such as promoters and the like.

The polynucleotide of interest is generally inserted into an expression vector, in which it is operatively linked to components which allow its expression to be regulated, in particular such as transcription promoters and/or terminators.

Such an expression vector may be in particular a plasmid, a phage or any type of recombinant virus.

Among the prokaryotic transformation vectors which are well known to those skilled in the art, mention may be made of the ZAP Lambda phage vector and the pBluescript plasmid (Stratagene). Other vectors which are suitable for the transformation of E. coli cells include pET expression vectors (Novagen) for example, pET11a, which contains the T7 promoter, the T7 terminator, the E. coli inducible Lac operon and the Lac repressor gene; and pET 12a-c, which contains the T7 promoter, the T7 terminator and the E. coli omPT secretion signal.

The vectors which are particularly preferred for the transfection of mammalian cells are vectors containing the cytomegalovirus (CMV) promoters such as pcDNA1 (Invitrogen), vectors containing the MMTV promoter such as pMAMNeo (Clontech) and pMSG (catalogue n° 27-4506-01 from Pharmacia) and vectors containing the SV40 promoter such as pSVβ (Clontech).

In the present invention, a promoter refers to a DNA segment which controls the transcription of DNA to which it is operatively linked. The promoter region includes specific sequences which are sufficient for recognition of the RNA polymerases, for binding and the initiation of transcription. In addition, the promoter region includes sequences which modulates this recognition, and the initiation of the binding and of the transcription of the RNA polymerase activity. As examples of promoters considered for use in the present invention, mention may be made of the SV40 promoter, the cytomegalovirus promoter, the mouse mammary tumor virus promoter (induced by steroids) and the Maloney murine leukemia virus promoter.

Vectors can be administered to the patient by any method that delivers materials to cells of the patient, such as by injection into the interstitial space of tissues such as muscles or skin, introduction into the circulation or into body cavities or by inhalation or insufflation. A naked polynucleotide may be injected or otherwise delivered to the animal with a pharmaceutically acceptable liquid carrier. For all applications, the liquid carrier is aqueous or partly aqueous, comprising sterile, pyrogen-free water. The pH of the preparation is suitably adjusted and buffered.

Antisense nucleic acids may be administered similarly.

Therapeutic Targets for Myelin Disorders

Identification of Ulip/CRMP proteins as mediators of myelin disorders can help characterizing new therapeutic targets such as endogenous agents that would for example specifically interact with Ulip/CRMP proteins in said pathological conditions.

The present invention thus provides a method for identifying endogenous agents as therapeutic targets for the prevention or the treatment of myelin disorders comprising:

contacting a cell, a tissue sample, a biological liquid sample, or an extract thereof, from a patient affected with a myelin disorder, with a Ulip/CRMP protein in a suitable medium allowing the Ulip/CRMP protein to interact with an endogenous agent;

determining if the Ulip/CRMP protein interacts with an endogenous agent;

identifying those endogenous agents interacting with the Ulip/CRMP protein as therapeutic targets for the prevention or the treatment of myelin disorders.

Preferably, said endogenous agent interacts with Ulip2/CRMP2 and/or Ulip6/CRMP5.

In the context of the present invention, a “cell extract” can be represented by a cell lysate or a cytosolic fraction for instance. Preferably, said cell is an oligodendrocyte.

A preferred embodiment for said tissue sample is a brain tissue sample. The biological liquid may be blood or spinal fluid in particular.

The following examples illustrate the invention without limiting the scope.

### EXAMPLES

Materials and Methods

Reagents.

Unless otherwise specified, all reagents were purchased from Sigma (L'Isle d'Abeau, France).

Production of Recombinant Proteins.

cDNAs coding for mouse Ulip1/CRMP4 (access number X87817), Ulip2/CRMP2 (access number Y10339), Ulip3/CRMP1 (access number Y09080), and Ulip4/CRMP3 (access number Y09079) were cloned in-frame with a flag sequence (Sigma) in the pSG5 vector (Stratagene, Amsterdam, The Netherlands) and used to produce recombinant proteins in HeLa cells. Human Ulip6/CRMP5 cDNA, cloned in-frame with the Lac-Z gene in pBluescript KS, was used to produce bacterial recombinant protein. Briefly, E. coli cells were grown for 1 h at 37° C., then Ulip6/CRMP5 expression was induced with IPTG (0.1 mM). After 3 h at 37° C., the cells were lysed by sonication and the soluble extract containing the Ulip6/CRMP5 recombinant protein obtained by centrifugation for 10 min at 2,000 g.

Antibodies.

The peptides chosen to generate specific antisera were KEMGTPLADTPTRPVTRHGG (SEQ ID n°1, amino acids 505-524) for anti-Ulip6/CRMP5, LEDGTLHVTEGS (SEQ ID n° 12) and ITGPEGHVLSRPEEVE (SEQ ID n°13) (amino acids 454-465 and 217-232, respectively) for anti-Ulip2/CRMP2, LTSFEKWHEMDTKS (SEQ ID n°14, amino acids 117-131) for anti-Ulip3/CRMP1, and EHDSHAQLRWRVL (SEQ ID n°15, amino acids 664-676) for anti-neuropilin-1. The synthetic peptides were conjugated to keyhole limpet hemocyanin and used to immunize rabbits or rats as previously described (Honnorat et al., 1999). The antibodies were purified from anti-Ulip6/CRMP5, anti-Ulip3/CRMP1, and anti-neuropilin-1 antisera using the corresponding immobilized peptide.

Protein Samples.

Male rats (OFA; Iffa-Credo, L'Arbresle, France) were anesthetized with pentobarbital. Tissues Were sonicated in 10 mM Tris-HCl, pH 7.4, 0.02% sodium azide, 1 mM EDTA, 0.2% Triton X-100, 10 μg/ml of leupeptin, 5 μg/ml of pepstatin, and 10 μg/ml of aprotinin, then centrifuged for 10 min at 2,000g at 4° C. The proteins in the supernatant were quantified (Coomassie Plus Protein Assay Reagent, Pierce, Interbiotech, Montluçon, France), diluted in the homogeneization buffer to a concentration of 2 mg/ml for neural tissues or 4 mg/ml for non-neural tissues, and stored at −20° C. until required.

Purified Oligodendrocyte Cultures.

Oligodendrocytes were isolated from six 4-week-old Sprague Dawley male rats (Iffa-Credo) using the procedure of Lisak et al. (1981), as modified by Lubetzki et al. (1988). Freshly isolated cells were plated on poly-L-lysine-coated glass coverslips (OSI, Maurepas, France) in 24-well plates (Costar Corporation, Cambridge, Mass.) at a density of 5×10⁴ cells/well, initially for 1 h in Dulbecco's modified Eagle's medium (DMEM; Life Technologies, Cergy-Pontoise, France) containing 10% fetal calf serum (FCS; Eurobio, Les Ulis, France) to facilitate attachment, then in standard culture medium consisting of Bottenstein and Sato medium (BS) (Bottenstein and Sato, 1979) supplemented with 5 U/ml of penicillin and 5pg/ml of streptomycin (Life Technologies).

RT-PCR Analysis.

Total cellular RNAs were extracted from the purified oligodendrocytes using RNA-zol B (Bioprobe, Montreuil sous Bois, France), according to the manufacturer's instructions. Reverse transcription was performed on 1 μg of total RNA using oligo-dT primers and Moloney murine leukemia virus reverse transcriptase (Life Technology, Cergy Pontoise, France). Ten percent of the RT product was used to perform the PCR. The following pairs of synthetic oligonucleotides were used as primers: 5′-ATAGACACGATGCCAMGACCTTACC-3 (SEQ ID n° 16) and 5′-ATTACCGCACCATCCTCAAGGC-3′ (SEQ ID n° 17) for CRMP1/Ulip3 (270 bp amplified cDNA fragment), 5′-T ATCACCCATCCCTTACTCTTCTGG-3′ (SEQ ID n° 18) and 5′-CAGMGAAAAAGCCAGAACAGACCG-3¹ (SEQ ID n° 19) for CRMP2/Ulip2 (141 bp amplified cDNA fragment), 5′-CCCCTCCCCATAAACTCTCTTTTGG-3′ (SEQ ID n° 20) and 5′-CTGGAAAGTTCACAGGCTGG-3′ (SEQ ID n° 21) for CRMP3/Ulip4 (200 bp amplified cDNA fragment), 5′-CCTACCAGGGCAAGAAGMCATTCC-3′ (SEQ ID n° 22) and 5′-CCGCMTGGTCTTCACACCTCC-3′ (SEQ ID no 23) for CRMP4/Ulip1 (173 bp amplified cDNA fragment), 5′-CTGTGGATGTGGACATGAAGC-3′ (SEQ ID n° 24) and 5′-AGCAATAAACAGGTGGMGGTC-3′ (SEQ ID n° 25) for proteolipid protein (PLP) an oligodendrocytic marker, (Monge et al., 1986), 5′-AGAGAGATTCGCACTCA-3′ (SEQ ID n° 26) and 5′-AGTGCCTCCTGGTAACTGG-3′ (SEQ ID n° 27) for glial fibrillary associated protein (GFAP), an astrocytic marker (Palfreyman et al., 1979), and 5′-GAAGAGTGGTTCAAGAGCCG-3′ (SEQ ID n° 28) and 5′-TGCCATCTTGACATTGAGGAGGTCC-3′ (SEQ ID n° 29) for the low molecular weight neurofilament protein (NF-L), a neuronal marker (Julien et al., 1987). The cDNA was denatured at 94° C for 5 minutes and then 35 cycles of PCR were carried out using Ampli-Taq DNA polymerase (Life Technology). The cycle profile consisted of denaturation at 94° C. for 45 s, annealing at 62° C. for 45 s, and elongation at 72° C. for 2 min. The PCR products were fractionated by electrophoresis on 1.8 agarose gels. Cyclophilin cDNA was used as an internal control (Danielson et al., 1988). The specificity of the assay was checked by sequencing the RT-PCR amplified fragments.

cDNA Cloning.

The cDNA library used in this study was a human spinal cord cDNA library in lambda gt11 phage (Clontech, Palo Alto, Calif., USA). Recombinant phages were screened at a density of 2×10⁴ PFU per 150-mm plate of E. coli Y1090r⁻. The library was first screened using serum from a patient with anti-CV2 antibodies (number: 94-799; Rogemond and Honnorat, 2000), primary antibody binding being visualized using peroxidase-labeled anti-human IgG antibody and colorimetric detection with diaminobenzidine. Positive clones were purified by several rounds of antibody screening until 100% of the plaques gave positive signals. Four positive clones were obtained, PCR-amplified, and sequenced. The longest (C97: 1.6 kb) was subcloned into the EcoR1 sites of pBluescript KS (Stratagene) and resequenced. To isolate the full-length cDNA, the human spinal cord cDNA library was screened using a ³²P-labeled 270 bp fragment of clone C97, obtained by PCR using primers chosen on the basis of the sequence of the partial cDNA clone (C97). Hybridization was performed using ExpressHyb™ hybridization solution (Clontech) and positive clones purified and sequenced. One of these, containing the complete coding region, was subcloned into pBluescript KS (Stratagene) and resequenced.

Northern Blot Analysis.

Northern blot analysis of Ulip6/CRMP5 expression was performed on a human adult multiple tissue RNA blot (MTN, Clontech) containing 2 μg of purified polyA⁺ RNA using the full-length cDNA (2 kb) labeled with alpha³²P-dCTP by random priming (Life Technologies). Hybridization was carried out in ExpressHyb™ hybridization solution (Clontech) following the manufacturer's instructions and the blot exposed to X-ray film at −80° C.

Western Blot Analysis.

Proteins were separated by SDS-PAGE and transferred to PVDF membranes (Millipore, St Quentin-en-Yvelines, France) using a semi-dry electroblotting system with a continuous buffer (Tris 25 mM, glycine 192 mM, methanol 20%, pH 8.5). The membranes were saturated with 2% non-fat dry milk in phosphate-buffered saline (PBS), then probed with primary antibodies. Bound antibodies were detected using peroxidase-coupled anti-IgG antibodies and diaminobenzidine oxidation. Immunohistochemistry. Four adult male, four 2-week-old (P15), four 5-day-old (P5), and four pregnant female rats (OFA; Iffa-Credo) were used. The adult male and P15 rats were anesthetized with pentobarbital and perfused intra-cardiacally with 4% paraformaldehyde in 0.1 M phosphate buffer, pH 7.4, (PB), then the brains were removed and postfixed in 4% paraformaldehyde for 12 h. The brains of anesthetized P5 rats were fixed by immersion for 12 h in 4% paraformaldehyde in PB. After 3 rinses and overnight incubation in PB/20% sucrose, the brains were frozen at −60° C in methyl-butane. 16- and 19-day embryos (E16 and E19, respectively) were removed from the anesthetized pregnant females and fixed by immersion for 12 h in 4% paraformaldehyde, then treated in the same way as the adult tissues. Sagittal cryostat sections (14 μm thick) were collected on Superfrost Plus slides (Polylabo, Strasbourg, France) and stored at −20° C. until required. Immunohistochemistry was performed as described previously (Honnorat et al., 1998). Briefly, the tissue sections were incubated overnight at room temperature with anti-Ulip6/CRMP5 antibodies (1/100 dilution) or anti-Ulip2/CRMP2 antibodies (1/50 dilution) and bound antibodies detected using fluorescein-conjugated anti-rabbit IgG antibodies.

In-situ hybridization. Sense or antisense digoxigenin-labeled riboprobes were generated by transcription of mouse Ulip2/CRMP2 cDNA (access number Y10339) and human Ulip6/CRMP5 cDNA (SEQ ID N°1) in pBluescript SK, using the T3 or T7 promoters and labeling with digoxigenin-UTP (Roche, Meylan, France), following the manufacturer's instructions. The human Ulip6/CRMP5 cDNA-derived riboprobe was suitable for hybridization with rat tissue sections because the sequence of this human riboprobe displays more than 90% homology with the corresponding rat sequence. Tissue sections were prepared as described above for immunohistochemistry, then treated with the sense and antisense riboprobes. For neuropilin-1, after 48 h of culture, purified oligodendrocytes were fixed in 4% paraformaldehyde, then subjected to in situ hybridization with digoxigenin-labeled oligonucleotide probes (antisense: CAGACATGTGATACCAGAAGGTCATGCAGT, SEQ ID n°30, from the neuropilin-1 sequence, access number D50086) as described previously (Giger et al., 1996).

Receptor Affinity Probes.

Alkaline phosphatase (AP) was fused to the amino terminus of Sema3A as previously described (Bagnard et al., 1998). In order to characterize Sema3A binding sites in highly purified oligodendrocytes in culture, the cells were incubated for 90 min with the AP-Sema3A recombinant protein in Hanks balanced salt solution (HBSS) supplemented with 20% FCS, washed 3 times in PBS, then fixed for 1 h in 4% paraformaldehyde. After one wash in PBS, endogenous phosphatases were heat-inactivated at 65° C. for 50 min, then the preparations were equilibrated for 20 min with AP buffer (100 mM Tris, 100 mM NaCl, and 5 mM MgCl₂, pH 9.5) and the bound AP-Sema3A visualized using a staining solution containing 34 mg/ml of Nitro-blue-tetrazolium and 18 mg/ml of 5-bromo-4-chloro-3-indolylphosphate (Roche) in AP buffer. Immunostaining with monoclonal Rip antibody, an oligodendrocyte marker (Friedman et al., 1989), was then used to visualize oligodendrocytes. The controls performed consisted of oligodendrocytes incubated in culture medium without recombinant protein or in the presence of an excess of untagged Sema3A.

Oligodendrocyte Process Extension Assay.

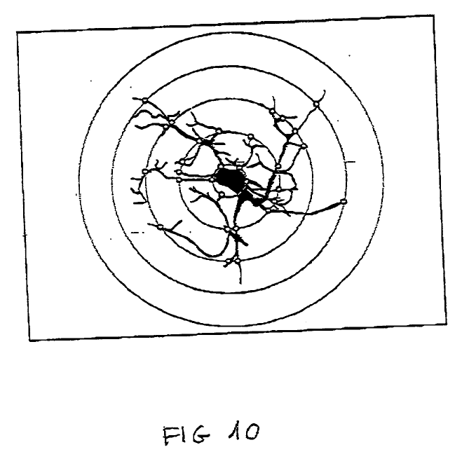

Highly purified mature oligodendrocytes were obtained and grown for 12 h in Bottenstein and Sato medium (BS, see above), then the BS medium was replaced with either Sema3A-conditioned medium (Sema3A medium) obtained from human embryonic kidney cells (HEK 293 cells) transfected with Sema3A expression vector, as described previously (Bagnard et al., 1998), or control medium from untransfected HEK 293 cells. Purified oligodendrocytes were also incubated for 48 h in Sema3A medium containing either 50 ng/ml of VEGF-165 (Miao et al., 1999) or various concentrations of antibodies (2, 4, or 8 pg/ml of immunopurified anti-neuropilin-1, anti-Ulip6/CRMP5, and anti-Ulip3/CRMP1 antibodies or 4, 8, or 20 μg/ml of IgG purified from anti-Ulip2/CRMP2 antisera and preimmune sera). The cultures were then fixed in 4% paraformaldehyde and analyzed. They were first immunostaining using the Rip monoclonal antibody and microphotographed using a X40 objective (Zeiss). Processes were quantified on the photographs using a grid composed of concentric circles separated by 10 μm and centered on the cell body (FIG. 10). The number of intersections between the circles and processes was counted for each cell, defining a branching index (BI); 20 cells were counted in each test sample to determine the mean BI. The results were confirmed in at least two independent experiments. Effects of treatments were quantified using the percentage extension compared to that under control conditions calculated as {(BI in control medium−BI in Sema3A medium)/BI in control medium}×100. The statistical significance of the results was evaluated using the unpaired Student t test.

Results

1) cDNA Cloning and Tissue Distribution of Human Ulip6/CRMP5.

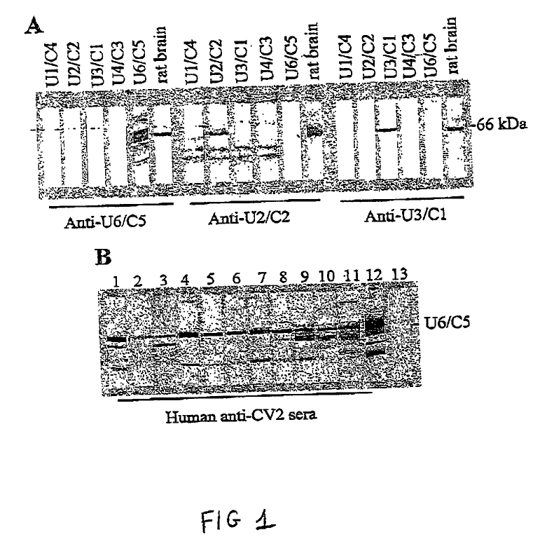

A human spinal cord cDNA library was screened using an anti-CV2 serum from a patient with PND and small cell lung carcinoma that recognized a 66 kDa protein on Western-blots of new-born rat brain protein extracts, but did not recognize any of the four previously known Ulip/CRMP recombinant proteins. This led to the identification of one partial-length clone (C97) containing a 1.6 kb cDNA insert yielding a 90 amino acid open reading frame which showed 35% homology with the C-terminal region of the four known human Ulip/CRMP proteins. The cDNA containing the full-length coding region was obtained by screening the same library with a radioactive probe corresponding to the coding region of C97 (270 bp). A 2 kb cDNA, referred to as Ulip6/CRMP5, that contains an open reading frame coding for 564 amino acids, was isolated (SEQ ID n°1). The C-terminal region of this protein was identical to the 90 amino acids encoded by C97. On Western blots, the Ulip6/CRMP5 recombinant protein was recognized by all 20 anti-CV2 sera tested (FIG. 1B), but not by 100 sera from patients without PND (half of them having small cell lung carcinoma), suggesting that Ulip6/CRMP5 was the major antigen recognized by anti-CV2 antibodies. The overall sequence of the Ulip6/CRMP5 cDNA consists of 3074 bp made up of a 162 bp 5′-non-coding region, a 1692 bp protein coding region, and a 1220 bp 3′-non-coding region. The initiation codon was assigned to the Met codon at position 163-165. The deduced protein sequence predicted a protein with a molecular mass of 61.424 kDa and an isoelectric point of 7.46.

Alignment of the sequence of the Ulip6/CRMP5 protein with those for the four known human Ulip/CRMP proteins showed 48-50% identity. Ulip6/CRMP5 and the other members of the family share the same degree of identity (about 33%) with the C. elegans gene product, unc-33 (Byk et al., 1998), a gene required for neurite outgrowth and axonal guidance (Li et al., 1992). The Ulip6/CRMP5 sequence contains consensus sites for several protein kinases, such as casein kinase 11 (8 sites), tyrosine kinase (2 sites), protein kinase A (1 site), and protein kinase C (8 sites). Alignment of the sequence of the human Ulip6/CRMP5 protein with those of rat CRAM (access number: AB029432) and mouse CRMP5 (access number: AF249295) showed 97% identity and comparison of the cDNA sequences showed more than 80% identity (more than 90% in the coding region).

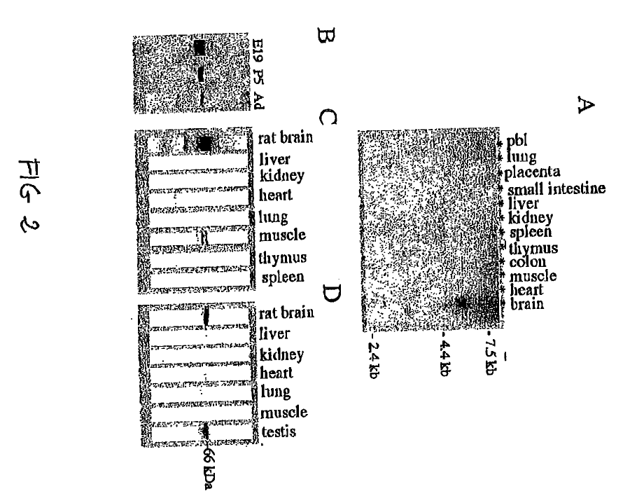

Northern blot analysis using a Ulip6/CRMP5 RNA probe identified a 5.5 kb band in human brain mRNA, while mRNAs prepared from various adult human peripheral tissues gave no hybridization signal (FIG. 2A), indicating preferential expression of Ulip6/CRMP5 mRNA in neural tissue. Expression of Ulip6/CRMP5 protein was analyzed by Western blotting using rabbit polyclonal antisera which, as shown in FIG. 1A, recognized the Ulip6/CRMP5 recombinant protein, but not the other four Ulip/CRMPs. As for the other Ulip/CRMPs (Hamajima et al., 1996; Wang and Strittmatter, 1996; Byk et al., 1998), Ulip6/CRMP5 protein was highly expressed in the embryonic brain and showed a dramatic downregulation during ontogenesis, as illustrated in the cerebellum (FIG. 2B). During development, Ulip6/CRMP5 was mainly detected in brain and lightly in muscle (P1, FIG. 2C). In adult rat tissue extracts, expression of Ulip6/CRMP5 was seen in brain and, at a lower level, in testis but not in muscle (FIG. 2D).

2) Distribution of Ulip6/CRMP5 in the Developing and Adult Rat Brain.

In order to investigate the function of Ulip6/CRMP5, the authors of the present invention determined the distribution pattern of the mRNA and protein using, respectively, in situ hybridization or immunohistochemistry on sections of E16 and E19 rat embryos and post-natal rat brain (P5, P15, and adult). Sense probes and pre-immune serum, used as controls, gave no signals (not shown). Ulip6/CRMP5 mRNA and protein were found to be highly expressed in the embryonic (E16 and E19) and post-natal (P5 and P15) brain and downregulated in the adult. The distribution of the protein was studied using anti-Ulip6/CRMP5 antibodies, which specifically recognized recombinant Ulip6/CRMP5 protein (FIG. 1A). The results are summarized in annexed Table 1 and described in detail below.

The observed distribution was identical to that previously described with anti-CV2 sera (Honnorat et al., 1996, 1998, 1999). In addition, the distribution of Ulip6/CRMP5 mRNA and protein in the adult brain was similar to the distribution of Ulip2/CRMP2, so Ulip6/CRMP5 and Ulip2/CRMP2 expression patterns were compared in detail in embryonic and post-natal rat brain.

a) Distribution of Ulip6/CRMP5 mRNA and Protein in the Developing Brain and Comparison with Ulip2/CRMP2 Expression

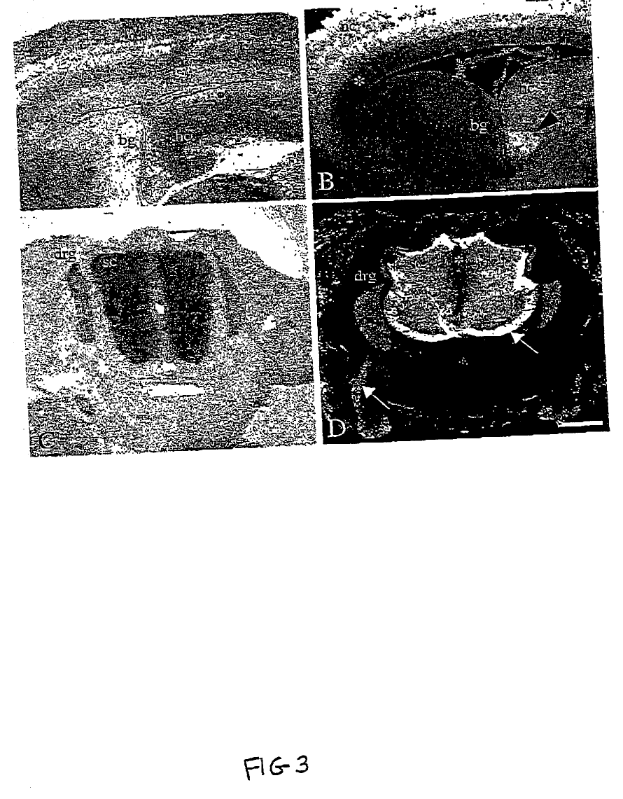

In the embryo and during the first post-natal days (P5), immunolabeling and in situ hybridization gave globally similar results (FIG. 3), indicating expression of Ulip6/CRMP5 protein in cells expressing mRNAs. All ventricular regions, such as in the cortex (FIGS. 3A and B) and spinal cord (FIGS. 3C and D), in which mitosis occurs, were always negative, suggesting that expression of Ulip6/CRMP5 mRNA and protein was restricted to postmitotic neural cells. At E16, E19, P5, and P15, Ulip6/CRMP5 expression was prominent in the neocortex, hippocampus, and spinal cord (FIG. 3, Table 1) and was also seen in the retina, hypothalamus, thalamus, midbrain, cerebellum, olfactory epithelium, olfactory bulb, and dorsal root ganglia (Table 1). Several neuronal fibers, such as those in the fimbria (FIG. 3B), spinal tracts or peripheral nerves (FIG. 3D) were also immunostained. The intensity of labeling of cell bodies and fibers decreased during the first two weeks after birth.

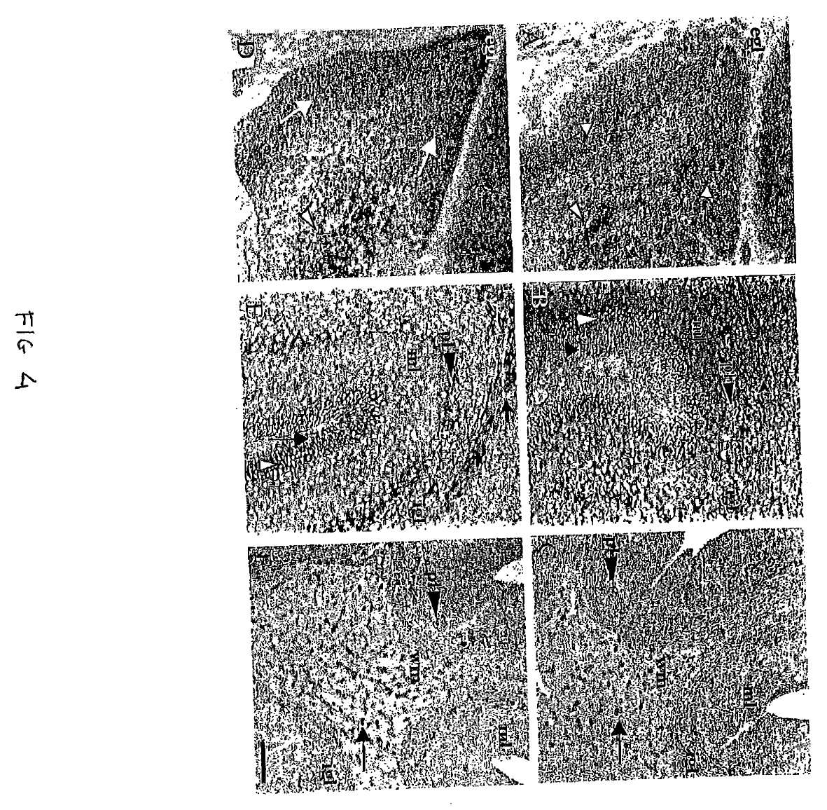

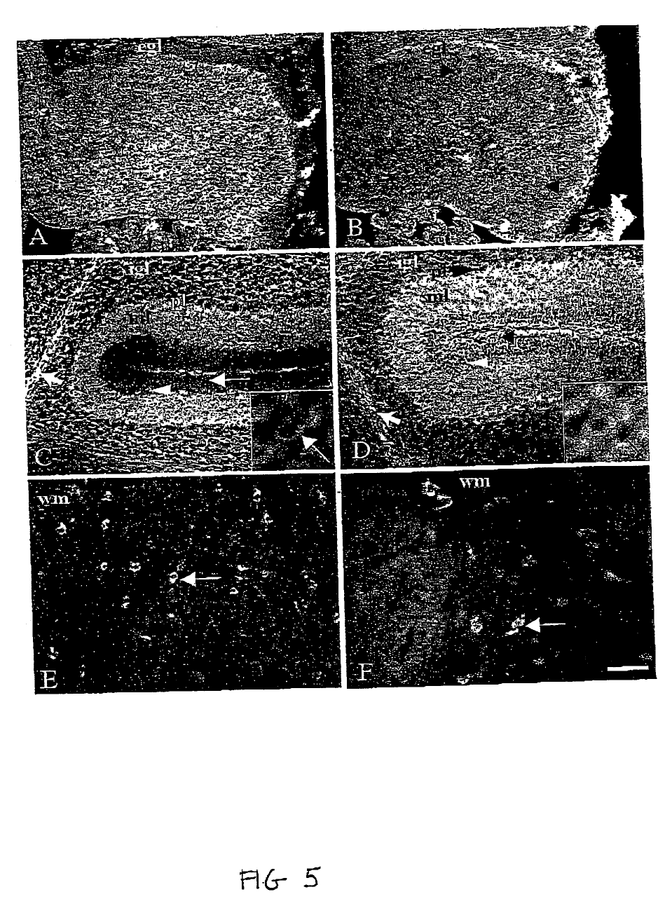

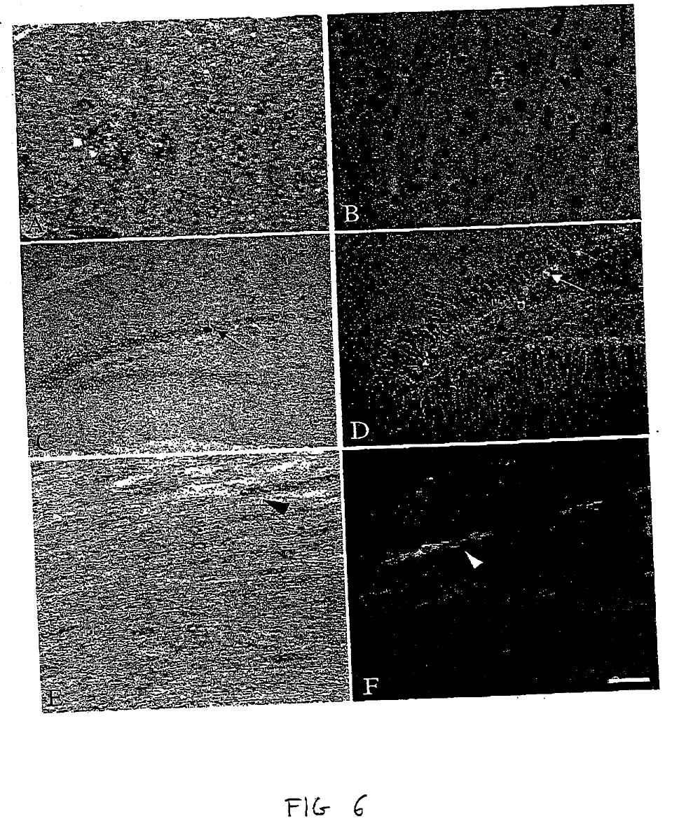

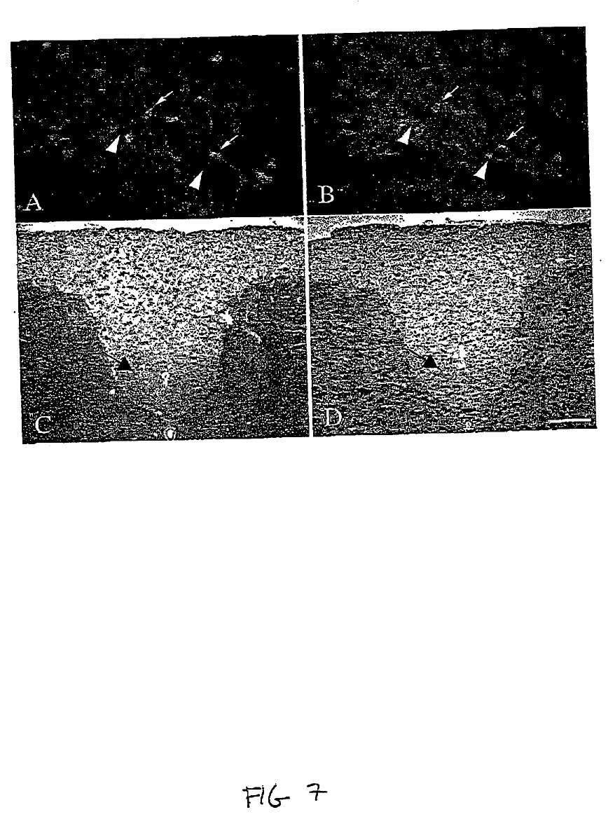

Temporal expression of Ulip6/CRMP5 and Ulip2/CRMP2 was compared in the developing cerebellum, chosen as a model structure characterized by postnatal directional migration, differentiation, and synaptogenesis with precise spatio-temporal order of positioning (Altman, 1972). At E19, Ulip6/CRMP5 mRNA and protein were expressed in all cerebellar layers, except the external granular layer (EGL) in which mitosis occur (FIGS. 4A and 5A), while Ulip2/CRMP2 mRNA and protein were highly expressed in the EGL and, to a lesser extent, in the inner part of the cerebellum (FIGS. 4D and 5B). At P5 and P15, Ulip6/CRMP5 was not expressed in the external part of the EGL, but was expressed in the internal part (FIGS. 4B and 5C) in which future granular neurons start migrating towards the internal granular layer (IGL), suggesting that Ulip6/CRMP5 is expressed by postmitotic granular neurons that are starting to migrate. At these stages, the neural progenitors in the external part of the EGL expressed high levels of Ulip2/CRMP2 but not Ulip6/CRMP5 (FIGS. 4E and 5D). Double-labeling showed that, in the internal part of the EGL, Ulip2/CRMP2 and Ulip6/CRMP5 proteins were co-expressed in granular neurons (FIGS. 5C and 5D inserts). At P15, Ulip6/CRMP5 mRNA and protein were also expressed by granular neurons in the IGL, but not by Purkinje cells (FIGS. 6B and 7C), whereas Ulip2/CRMP2 mRNA and protein were highly expressed in Purkinje cells, but only weakly detectable in the granular neurons of the IGL (FIGS. 4E and 5D). At P15, Ulip2/CRMP2 and Ulip6/CRMP5 proteins were both highly expressed in growing fibers of the molecular layer and white matter (FIGS. 5C and D).

b) Distribution of Ulip6/CRMP5 mRNA and Protein in the Adult Brain and Comparison with Ulip2/CRMP2 Expression

Temporal expression of Ulip6/CRMP5 and Ulip2/CRMP2 was compared in the developing cerebellum, chosen as a model structure characterized by postnatal directional migration, differentiation, and synaptogenesis with precise spatio-temporal order of positioning (Altman, 1972). At E19, Ulip6/CRMP5 mRNA and protein were expressed in all cerebellar layers, except the external granular layer (EGL) in which mitosis occur, while Ulip2/CRMP2 mRNA and protein were highly expressed in the EGL and, to a lesser extent, in the inner part of the cerebellum. At P5 and P15, Ulip6/CRMP5 was not expressed in the external part of the EGL, but was expressed in the internal part in which future granular neurons start migrating towards the internal granular layer (IGL), suggesting that Ulip6/CRMP5 is expressed by postmitotic granular neurons that are starting to migrate. At these stages, the neural progenitors in the external part of the EGL expressed high levels of Ulip2/CRMP2 but not Ulip6/CRMP5. Double-labeling showed that, in the internal part of the EGL, Ulip2/CRMP2 and Ulip6/CRMP5 proteins were co-expressed in granular neurons. At P15, Ulip6/CRMP5 mRNA and protein were also expressed by granular neurons in the IGL, but not by Purkinje cells, whereas Ulip2/CRMP2 mRNA and protein were highly expressed in Purkinje cells, but only weakly detectable in the granular neurons of the IGL. At P15, Ulip2/CRMP2 and Ulip6/CRMP5 proteins were both highly expressed in growing fibers of the molecular layer and white matter.

Between P20 and the adult, the pattern of expression of Ulip6/CRMP5 was constant. In the adult brain, neurons expressing Ulip6/CRMP5 were identified by their anatomical localization, size and shape. Ulip6/CRMP5 mRNA and protein were expressed in migrating neurons in the rostral migratory stream of the olfactive bulb, scarce neurons throughout the neocortex, and granular neurons in the juxta-hilar portion of the granular cell layer of the hippocampus. Moreover, low expression of Ulip6/CRMP5 mRNA in the absence of detectable protein was seen in a few neurons, namely the molecular and granular neurons of the IGL and a few Purkinje cells in the cerebellum. Similarly, Ulip2/CRMP2 mRNA was expressed in Purkinje cells and, to a lesser extent, in molecular and granular neurons of the IGL, despite the absence of detectable Ulip2/CRMP2 protein in these neurons. The presence of Ulip6/CRMP5 and/or Ulip2/CRMP2 mRNAs in some neurons in the absence of detectable protein indicates either rapid turnover of the protein or translational or post-translational regulation of the protein. Phosphorylation, glycosylation and/or association of Ulip6/CRMP5 and Ulip2/CRMP2 with other proteins (Wang and Strittmatter, 1997; Bulliard et al., 1997; Inatome et al., 2000) could limit the recognition of the protein by the antibodies.

3) Ulip/CRMP proteins are expressed in oligodendrocytes

a) In situ Hybridization and Immunohistochemistry Analysis of Ulip6/CRMP5 and Ulip2/CRMP2 Oligodendrocyte Expression

In the adult brain, the strongest Ulip6/CRMP5 mRNA and protein expression was seen in oligodendrocytes of the myelinated tracts of the spinal cord, hindbrain, midbrain, and cerebellum. Ulip6/CRMP5 mRNA and protein were detected in small cells distributed in rows in the myelinated tracts and double-labeled with the oligodendrocyte-specific Rip monoclonal antibody as previously described using anti-CV2 sera (Honnorat et al., 1996, 1998). Ulip6/CRMP5-expressing oligodendrocytes were detected according to an increasing rostral to caudal gradient, starting in the anterior part of the basal cerebral peduncle. In the brainstem, the highest number of Ulip6/CRMP5-positive oligodendrocytes was found in the cerebellar peduncles, the spinal tract of the trigeminal nerve, the tractus pyramidalis and the ventro-spino-cerebellar tract. Within the nerve tracts, immunostained cells were widespread and bore thin stained processes clinging to the myelin sheath. The spinal cord contained the greatest number of immunostained cells. All along the spinal cord, many Ulip6/CRMP5-positive oligodendrocytes were seen in all the tracts of the white matter, except in the ventral part of the dorsal corticospinal tract, while no labeling was seen in the gray matter. These immunostained cells defined a subset of oligodendrocytes estimated, using anti-CV2 sera, to account for one third of spinal cord oligodendrocytes, with an rostro-caudal gradient (Honnorat et al., 1998). Ulip6/CRMP5-positive oligodendrocytes were rarely found in the forebrain: the gray matter or myelinated fiber tracts, such as the corpus callosum or anterior commissure.

Similarly, Ulip2/CRMP2 has been shown to be expressed by a subpopulation of oligodendrocytes in adult brain. In spinal cord, hindbrain and midbrain white matter, all oligodendrocytes stained by anti-Ulip6/CRMP5 antibodies were double-stained by anti-Ulip2/CRMP2 antibodies, demonstrating that these two Ulip/CRMP proteins were coexpressed by certain oligodendrocytes. Interestingly, some Ulip2/CRMP2 protein-expressing oligodendrocytes in the midbrain and spinal cord, i.e. the ventral part of the dorsal cortico-spinal tracts, did not express Ulip6/CRMP5. As Ulip2/CRMP2 protein is expressed by only 40% of spinal cord oligodendrocytes, three different subsets of oligodendrocytes can be distinguished in the spinal cord, one expressing both Ulip6/CRMP5 and Ulip2/CRMP2, another expressing only Ulip2/CRMP2, and a third expressing neither. On the other hand, it is noteworthy that, during ontogenesis, Ulip2/CRMP2 was detectable in oligodendrocytes at P15, while the earliest Ulip6/CRMP5-expressing oligodendrocytes appeared at P18.

b) Expression Ulip3/CRMP1, Ulip2/CRMP2, Ulip4/CRMP3, and Ulip 1/CRMP4 in Purified Oligodendrocytes

Using primers specific for each of the four Ulip/CRMP transcripts (Ulip3/CRMP1, Ulip2/CRMP2, Ulip4/CRMP3, and Ulip1/CRMP4), RT-PCR analysis were performed on RNA extracted from highly purified (90-95% GalC-positive) adult brain oligodendrocytes. All four Ulip/CRMP members were detected in the oligodendrocyte preparation. Amplified cyclophilin and PLP mRNAs were detected in each preparation under the same conditions. In contrast, no signal was detected using either the neurone-specific NF-L primers or the astrocytes-specific GFAP primers, confirming the high degree of purity of the oligodendrocyte preparation.

Ulip6/CRMP5 expression in highly purified oligodendrocytes was demonstrated with Ulip6/CRMP5 immunolabeling in oligodendrocytes double labeled with RIP.

4) Inhibition of Oligodendrocyte Process Extension by Sema3A Involvement of Ulip6/CRMP5 and Ulip2/CRMP2.

To investigate the role of Ulip6/CRMP5 and Ulip2/CRMP2 in oligodendrocytes, the authors used highly purified adult rat brain oligodendrocytes.

Since Ulip2/CRMP2 is considered as a mediator of the Sema3A-induced axon collapse (Goshima et al., 1995), the effect of Sema3A on adult brain oligodendrocytes was studied. The presence of Sema3A-binding sites on these cells was demonstrated using a receptor affinity probe, alkaline phosphatase-Sema3A (AP-Sema3A) fusion protein (Bagnard et al., 1998). Specific AP-Sema3A binding was detected on all oligodendrocytes (cell bodies and processes) by double immunostaining using Rip monoclonal antibody. No staining was seen when AP was used instead of AP-Sema3A or in the presence of a large excess of untagged Sema3A.

The presence of neuropilin-1, a component of the Sema3A receptor complex (He and Tessier-Lavigne, 1997; Kolodkin, 1997), was shown by in situ hybridization using neuropilin-1 probes. All Rip-positive oligodendrocytes were labeled by neuropilin-1 antisense probes, while no cells were labeled by sense probes.

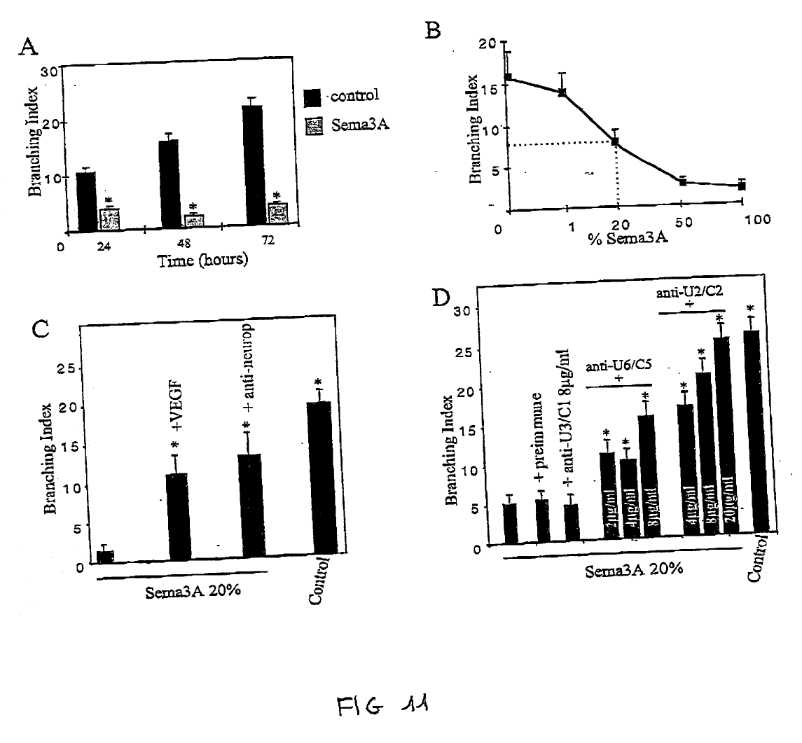

Since the cultured oligodendrocytes had been shown to have Sema3A binding sites and to express the neuropilin-1 receptor, oligodendrocytes response to soluble Sema3A was examined by incubating them for 24, 48, or 72 h with conditioned medium from untransfected or Sema3A-expressing HEK 293 cells. When cultured in the control medium, the cells displayed the morphological characteristics of oligodendrocytes, having round or ovoid cell bodies with a radiating array of thin tapering and branching processes, and expressing the oligodendrocyte marker, Rip; under these conditions, the oligodendrocytes could survive up to 20 days in culture. After 24 h incubation in a Sema3A-conditioned medium, the oligodendrocytes showed significant loss of processes compared with controls. To quantify oligodendrocyte arborization, we used a grid of concentric circles separated by 10 μm centered on the cell body and counted the number of intersections between the circles and the oligodendrocyte processes (FIG. 10), defining a branching index (BI). Freshly isolated purified oligodendrocytes initially had a mean BI close to zero, then started to spontaneously send out processes with the time course shown in FIG. 11A (control), with a maximal mean BI of 21.5 at 72 h of culture. In Sema3A-conditioned medium, the BI decreased by 72% at 24 h, 81% at 48 h, and 88% at 72 h compared with controls (p<0.0001) (FIG. 11A). The Sema3A dose-response curve, determined using a range of dilutions of Sema3A-conditioned medium (undiluted to 1/100) diluted in control medium (FIG. 11B), showed a sigmoid shape consistent with a specific biologic effect. The half-effect, corresponding to a BI reduction of 50%, (p<0.005), was obtained at a 1/20 dilution (25 ng/ml of Sema3A; Bagnard et al., 1998). The Sema3A effect seen after 24 h incubation was totally reversed after removal of the Sema3A-conditioned medium and 72 h incubation in control medium, the mean BI increasing to 20.8. It is noteworthy that oligodendrocytes cultured in Sema3A-containing medium expressed Rip, a marker of late stages of oligodendrocytic differentiation (Friedman et al., 1989).

The effect of Sema3A signal on oligodendrocyte process extension was further investigated by blocking neuropilin-1 using antibodies directed against the MAM part of the receptor (Chen et al., 1998) which have been successfully used to block the effect of Sema3A on neurons. After 48 h incubation in Sema3A-conditioned medium in the presence of anti-neuropilin-1 antibodies (4 pg/ml), the oligodendrocytes displayed a BI reduction of 25% compared with a reduction of 81% in the absence of antibodies (p<0.001) (FIG. 11C). Furthermore, when VEGF-1 65, which has been proposed to antagonize Sema3A binding to neuropilin-1 (Miao et al., 1999), was added to Sema3A-conditioned medium at a concentration of 50 ng/ml, the BI was reduced by only 40% compared with 81% in the absence of VEGF-165 (p<0.001) (FIG. 11C). These results indicated that the effect of Sema3A on oligodendrocytes was mediated by neuropilin-1.

To assess the role of Ulip2/CRMP2 and/or Ulip6/CRMP5 in transducing the Sema3A-induced inhibition of oligodendrocyte process extension, anti-Ulip2/CRMP2 antibodies were used to block Ulip2/CRMP2, as described by Goshima et al. (1995), and anti-Ulip6/CRMP5 or anti-CV2 antibodies to block Ulip6/CRMP5. After 48 h incubation in Sema3A medium containing anti-Ulip2/CRMP2 antibodies at different concentrations (4, 8, and 20 μg/ml), a dose-dependent increase in the mean BI (BI =14.2 at 8 μg/ml) was seen compared to oligodendrocytes grown in Sema3A-conditioned medium in the absence of antibodies (BI=5, p<0.001) (FIG. 11D). A significant block of the Sema3A effect on oligodendrocyte process extension was also seen using anti-Ulip6/CRMP5 antibodies (2, 4, and 8 pg/ml) (FIG. 11D) and anti-CV2 antibodies. In contrast, anti-Ulip3/CRMP1 antibodies, recognizing specifically the Ulip3/CRMP1 recombinant protein, or pre-immune sera had no effect (FIG. 11D). These results indicated that Ulip2/CRMP2 and Ulip6/CRMP5 mediate the Sema3A effect on oligodendrocyte process extension.

5) Inhibition of Oligodendrocyte Process Extension by Sema4D

The use of Semaphorin 4D on differentiated oligodendrocytes also dramatically reduces the process extensions and leads to a gradual disappearance of oligodendrocytes. In addition, antibodies against Sema4D block the death of undifferentiated oligodendrocyte progenitors (Dev cell line; Bagnard et al., 2001) induced by Sema4D expressing T lymphocytes. These data indicate that several Semaphorins may be able of modulating oligodendrocyte death via CRMP members.

Since Sema4D is expressed in CNS infiltrating lymphocytes, Sema4D could be implicated via the Ulip/CRMP proteins in the demyelinating neuro-inflammatory diseases.

Conclusion:

In the adult brain, the most intense Ulip6/CRMP5 in situ hybridization and immunohistochemistry labelings were seen in oligodendrocytes in the pons, cerebellum and spinal cord, a distribution similar to that seen for Ulip2/CRMP2, suggesting the coexpression of the two proteins.

Interestingly, similar coexpression or lack of coexpression of Ulip2/CRMP2 and Ulip6/CRMP5 were seen during brain development. In the cerebellum, only Ulip2/CRMP2 was highly expressed in the external part of EGL containing the mitotic neural precursors, while both Ulip2/CRMP2 and Ulip6/CRMP5 were expressed in the internal part of the EGL, which contains the postmitotic migrating neuronal precursors. After migration, neuronal precursors in the IGL showed high expression of Ulip6/CRMP5, but only low expression of Ulip2/CRMP2. In addition, during brain development, Ulip2/CRMP2 was expressed before Ulip6/CRMP5 in oligodendrocytes. Taken together, these results indicate that Ulip2/CRMP2 and Ulip6/CRMP5 may either have different roles in the intracellular signal cascade pathway in response to the same signal or mediate different signals, involved in the balance of positive and negative growth cues required in the regulation of neuronal migration /axonal growth and oligodendrocyte migration/process extension.

-o-o-o-o-o

All references cited in the present specification are incorporated in their entirety.

### REFERENCES

Altman J (1972) Postnatal development of the cerebellar cortex in the rat. I. The external germinal layer and the transitional molecular layer. J Comp Neurol 145:353-397

Altman J (1972) Postnatal development of the cerebellar cortex in the rat. II. Phases in the maturation of Purkinje cells and of the molecular layer. J Comp Neurol 145:399-463

Altman J (1972) Postnatal development of the cerebellar cortex in the rat. III. Maturation of the components of the granular layer. J Comp Neurol 145:465-513

Bagnard D, Lohrum M, Uziel D, Püschel A W, Bolz J (1998) Semaphorins act as attractive and repulsive guidance signals during the development of cortical projections. Development 125:5043-5053

Bagnard et al. (2001). J. Neurosc. 21:3332-3341.

Bottenstein J E, Sato G H (1979) Growth of a rat neuroblastoma cell line in serum-free supplemented medium. Proc Natl Acad Sci USA 76:514-517

Bulliard C, Zurbriggen R, Tornare J, Faty M, Dastoor Z, Dreyer J L (1997) Purification of a dichlorophenol-indophenol oxidoreductase from rat and bovine synaptic membranes: tight complex association of a glyceraldehyde-3-phosphate dehydrogenase isoform, TOAD64, enolase-gamma and aldolase C. Biochem J 324:555-563

Byk T, Ozon S, Sobel A (1998) The Ulip family phosphoproteins. Eur J Biochem 254:14-24

Chen H, He Z, Bagri A, Tessier-Lavigne M (1998) Semaphorin-neuropilin interactions underlying sympathetic axon responses to class III semaphorins. Neuron 21:1283-1290

Friedman B, Hockfield S, Black J A, Woodruff K A, Waxman S G (1989) In situ demonstration of mature oligodendrocytes and their processes: an immunocytochemical study with a new monoclonal antibody, rip. Glia 2:380-390.

Fukada M, Watakabe I, Yuasa-Kawada J, Kawachi H, Kuroiwa A, Matsuda Y, Noda M (2000) Molecular characterization of CRMP5, a novel member of the collapsin response mediator protein family. J Biol Chem (in press)

Giger R J, Wolfer D P, De Wit G M, Verhaagen J (1996) Anatomy of rat semaphorin III/collapsin-1 mRNA expression and relationship to developing nerve tracts during neuroembryogenesis. J Comp Neurol 375:378-392

Goshima Y, Nakamura F, Strittmatter P, Strittmatter, S M (1995) Collapsin-induced growth cone collapse mediated by an intracellular protein related to UNC-33. Nature 376:509-514

Gu Y, Hamajima N, Ihara Y (2000) Neurofibrillary tangle-associated collapsin response mediator protein-2 (CRMP-2) is highly phosphorylated on Thr-509, Ser-518, and Ser-522. Biochemistry 39:4267-4275

Gu Y, Ihara Y(2000) Evidence that collapsin response mediator protein-2 is involved in the dynamics of microtubules. J Biol Chem 275:17917-17920.

Hamajima N, Matsuda K, Sakata S, Tamaki M, Nonaka, M (1996) A novel gene family defined by human dihydropyrimidinase and three related proteins with differential tissue distribution. Gene 180:157-163

He Z, Tessier-Lavigne M (1997) Neuropilin is a receptor for the axonal chemorepellent Semaphorin Ill. Cell 90:739-751

Honnorat J, Antoine J C, Derrington E A, Aguera M, Belin, M F (1996) Antibodies to a subpopulation of glial cells and a 66 kD developmental protein in patients with paraneoplastic neurological syndromes. J Neurol Neurosurg Psychiatry 61:270-278

Honnorat J, Aguera M, Zalc B, Goujet C, Quach T, Antoine J C, Belin M F (1998) POP66, a paraneoplastic encephalomyelitis-related antigen, is a marker of adult oligodendrocytes. J Neuropathol Exp Neurol 57:311-322

Honnorat J, Byk T, Kuster I, Aguera M, Ricard D, Rogemond V, Quach T, Aunis D, Sobel A, Mattei M G, Kolattukudy P, Belin M F, Antoine, J C. (1999) Ulip/CRMP proteins are recognized by autoantibodies in paraneoplastic neurological syndromes. Eur J Neurosci 1 1:4226-4232

Köhler et Milstein, Nature, (1975), vol. 256, pp 495-497

Inatome R, Tsujimura T, Hitomi T, Mitsui N, Hermann P, Kuroda S, Yamamura H, Yanagi S (2000) Identification of CRAM, a novel unc-33 gene family protein that associates with CRMP3 and protein-tyrosine kinase(s) in the developing rat brain. J Biol Chem 275:27291-27302

Jayasena S. D. (1999) Aptamers: an emerging class of molecules that rival antibodies in diagnostics. Clinical Chemistry 45(9):1628-1650.

Kamata T, Subleski M, Hara Y, Yuhki N, Kung H, Copeland NG, Jenkins NA, Yoshimura T, Modi, W, Copeland, TD (1998). Isolation and characterization of a bovine neural specific protein (CRMP-2) cDNA homologous to unc-33, a C. elegans gene implicated in axonal outgrowth and guidance. Mol Brain Res 54:219-236

Kolodkin A L, Levengood D V, Rowe E G, Tai Y T, Giger R J, Ginty D D (1997) Neuropilin is a semaphorin III receptor. Cell 90:753-762

Li W, Herman R K, Shaw E (1992) Analysis of the Caenorhabditis elegans axonal guidance and outgrowth gene unc-33. Genetics 132:675-689 Lisak R P, Pleasure D E, Silberberg D H, Manning M C, Saida T (1981) Long term culture of bovine oligodendroglia isolated with a Percoll gradient. Brain Res 223:107-122

Lubetzki C, Gansmuller A, Lachapelle F, Lombrail P, Gumpel M (1988) Myelination by oligodendrocytes isolated from 4-6-week-old rat central nervous system and transplanted into new-born shivered brain. J Neurol Sci 88:161-175

Miao H Q, Soker S, Feiner L, Alonso J L, Raper J A, Klagsbrun M (1999) Neuropilin-1 mediates collapsin-1/semaphorin III inhibition of endothelial cell motility: functional competition of collapsin-1 and vascular endothelial growth factor-165. J Cell Biol 146:233-242

Minturn J E, Fryer H J L, Geschwind D H, Hockfield S (1995) TOAD-64, a gene expressed early in neuronal differentiation in the rat, is related to unc-33, a C. elegans gene involved in axon outgrowth. J Neurosci 15:6757-6766

Nacher J, Rosell D R, McEwen B S (2000) Widespread expression of rat collapsin response-mediated protein 4 in the telencephalon and other areas of the adult rat central nervous system. J Comp Neurol 424:628-639 Pasterkamp RJ, De Winter F, Holtmaat A J G D, Verhaagen J (1998). Evidence for a role of the chemorepellent semaphorin III and its receptor neuropilin-1 in the regeneration of primary olfactory axons. J Neurosci 23:9962-9976

Quach T T, Rong Y, Belin, M F, Duchemin A M, Akakoa H, Ding S, Baudry M, Kollattukudy P E, Honnorat J (1997) Molecular cloning and expression of a new unc-33 like cDNA from rat brain and its relation to paraneoplastic neurological syndromes. Mol Brain Res 46:329-332

Rogemond V, Honnorat J (2000) Anti-CV2 autoantibodies and paraneoplastic neurological syndromes. Clin Rev Allergy Immunol 19:48-56

Sambrook et al., (1989) Molecular Cloning, a laboratory Manual,, 9.47-9.62

Tuerk C. and Gold L., (1990) Systematic evolution of ligands by exponential enrichment: RNA ligands to bacterophage T4 DNA polymerase. Science 249:505-510.

Wang L H, Strittmatter S M (1996) A family of rat CRMP genes is differentially expressed in the nervous system. J Neurosci 16:6197-6207

Wang LH, Strittmatter SM (1997) Brain CRMP forms heterotetramers similar to liver dihydropyrimidinase. J Neurochem 69:2261-2269

Yoshida H, Watanabe A, Ihara Y (1998) CRMP-2 is associated with neurofibrillary tangles in Alzeimer's disease. J Biol Chem 273:9761-9768

What we claim is:

1. A method for the prevention or treatment of myelin disorders, comprising modulating a Ulip/CRMP activity.

2. The method of claim 1, wherein said Ulip/CRMP is Ulip6/CRMP5.

3. The method of claim 1, wherein said Ulip/CRMP is the Ulip2/CRMP2.

4. The method of claim 1, wherein the myelin disorder is multiple sclerosis.

5. The method of claim 1, wherein the myelin disorder is HTLV-1 associated myelopathy.

6. A method for the prevention or treatment of myelin disorders, comprising administering to a patient in need of such treatment a therapeutically effective amount of an agent selected from the group consisting of a Ulip/CRMP protein, a nucleic acid coding for a Ulip/CRMP protein, an anti-sense sequence capable of specifically hybridizing with said nucleic acid, an antibody directed against the Ulip/CRMP protein, and an aptamer capable of binding said protein, and a pharmacologically acceptable carrier.

7. The method of claim 6, wherein said Ulip/CRMP is Ulip6/CRMP5.

8. The method of claim 6, wherein said Ulip/CRMP is the Ulip2/CRMP2.

9. The method of claim 6, wherein the myelin disorder is multiple sclerosis.

10. The method of claim 6, wherein the myelin disorder is HTLV-1 associated myelopathy.

11. The method of claim 6, wherein said Ulip/CRMP is the Ulip6/CRMP5 protein which comprises the amino acid sequence SEQ ID n° 2.

12. The method of claim 6, wherein said Ulip/CRMP is the Ulip2/CRMP2 protein which comprises the amino acid sequence SEQ ID n° 4.

13. The method of claim 6, wherein said nucleic acid is the nucleic acid coding for the Ulip6/CRMP5 protein which comprises the nucleic acid sequence from nucleotides 163 to 1854 in SEQ ID n° 1.

14. The method of claim 6, wherein said nucleic acid is the nucleic acid coding for the Ulip2/CRMP2 protein which comprises the nucleic acid sequence from nucleotides 72 to 1790 in SEQ ID n° 3.

15. The method of claim 6 wherein said Ulip/CRMP protein is a purified Ulip6/CRMP5.

16. The method of claim 6 wherein said Ulip/CRMP protein is a purified Ulip2/CRMP2.

17. A method of diagnosing a myelin disorder in a subject, comprising:
evaluating the level of expression of at least one agent selected from the group consisting of a Ulip/CRMP protein and antibodies to a Ulip/CRMP protein present in the sample in a biological sample from said subject;
comparing the level of expression of said agent in the biological sample with expression levels of said agent in control subjects.

18. The method of claim 17, wherein the Ulip/CRMP protein is Ulip2/CRMP2 and/or Ulip6/CRMP5.

19. The method of claim 17, wherein the antibodies are antibodies to a Ulip2/CRMP2 protein and/or to a Ulip6/CRMP5 protein.

20. The method of claim 17, wherein the myelin disorder is multiple sclerosis.

21. The method of claim 17, wherein the myelin disorder is HTLV1-associated myelopathy.

22. A method for identifying agents useful for the prevention or treatment of myelin disorders, comprising
contacting a Ulip/CRMP protein or a Ulip/CRMP expressing cell with a test compound
determining if the test compound has a modulatory effect on the Ulip/CRMP activity; and
identifying those test compounds having a stimulatory or inhibitory effect on the Ulip/CRMP protein, as useful for the prevention or treatment of myelin disorders.

23. The method of claim 22 wherein said modulatory effect of the test compound is assessed by evaluating the level of expression of the Ulip/CRMP protein.

24. The method of claim 22 wherein said Ulip/CRMP expressing cell is an oligodendrocyte.

25. The method of claim 24 wherein said modulatory effect of the test compound is assessed by an oligodendrocyte process extension assay

26. The method of claim 22 wherein said Ulip/CRMP protein is a Ulip2/CRMP2 and/or a Ulip6/CRMP5 protein.

27. A Ulip/CRMP activity modulatory agent identified by the method of claim 22.

28. A composition for treating or preventing a myelin disorder comprising an agent according to claim 27 and a pharmaceutically acceptable carrier.

29. A method of treating or preventing a myelin disorder comprising administering to a patient in need of such treatment a therapeutically effective amount of the composition of claim 28.

30. A method for identifying agents useful for the prevention or treatment of myelin disorders, comprising:
contacting a Ulip/CRMP protein an inducer or effector of said protein with a test compound in a suitable medium allowing the interaction between the Ulip/CRMP protein and its inducer or effector protein;
determining if the test compound has a stimulatory or inhibitory effect on the interaction between the Ulip/CRMP protein and its inducer or effector protein; and
identifying those test compounds having a stimulatory or inhibitory effect on the interaction between the Ulip/CRMP protein and its inducer or effector protein, as useful for the prevention or treatment of myelin disorders.

31. The method of claim 30, wherein the Ulip/CRMP protein is a Ulip2/CRMP2 protein or a Ulip6/CRMP5 protein.

32. An agent capable of stimulating or inhibiting the interaction between a Ulip/CRMP protein and its inducer or effector protein identified by the method of claim 30.

33. An agent capable of stimulating or inhibiting the interaction between a Ulip6/CRMP5 protein and a Ulip2/CRMP2 protein identified by the method of claim 30

34. A composition for treating or preventing a myelin disorder comprising an agent according to claim 32 and a pharmaceutically acceptable carrier.

35. A composition for treating or preventing a myelin disorder comprising an agent according to claim 33 and a pharmaceutically acceptable carrier.

36. A method of treating or preventing a myelin disorder comprising administering to a patient in need of such treatment a therapeutically effective amount of the composition of claim 34.

37. A system for determining which compounds may be useful for treating or preventing a myelin disorder comprising
a testing means which allows one to contact a test compound and
a determining means to determine if the test compound has a stimulatory or inhibitory activity on the Ulip/CRMP protein, said activity being indicative of a compound potentially useful for treating or preventing a myelin disorder.

38. The system according to claim 37, wherein the Ulip/CRMP protein is a Ulip2/CRMP2 protein and/or a Ulip6/CRMP5 protein.

39. A method for identifying an endogenous agent as a therapeutic target for the prevention or the treatment of myelin disorders comprising:
contacting a cell, a tissue sample, a biological liquid sample, or an extract thereof, from a patient affected with a myelin disorder, with a Ulip/CRMP protein in a suitable medium allowing the Ulip/CRMP protein to interact with an endogenous agent;
determining if the Ulip/CRMP protein interacts with an endogenous agent;
identifying those endogenous agents interacting with the Ulip/CRMP protein as therapeutic targets for the prevention or the treatment of myelin disorders.

40. The method of claim 39 wherein the Ulip/CRMP protein is Ulip2/CRMP2 and/or Ulip/CRMP5.

41. The method of claim 39 wherein said cell is an oligodendrocyte.

42. The method of claim 39 wherein tissue sample is a brain tissue sample.

43. The method of claim 39 wherein biological liquid is blood or spinal fluid.


SEQUENCE LISTING (30 sequences)

SEQ ID NO: 1 - DNA, 3074 bp, Homo sapiens
cccgccccactctggactcccgcgctgggcgcgctgaggcggcccccgagcgagcgcgcgtgcagccgccgccgccccgagcacccgcagctccggcgccgcggcgagacggagacggaccgagccacgggcccccgcggccgcagcatctcggaggagaacatgcttgccaactcagccagcgtgaggatcctcatcaagggaggcaaggtggtgaacgatgactgcacccacgaggctgacgtctacatcgagaatggcatcatccagcaggtgggccgcgagctcatgatccctggcggggccaaggtgattgatgccacaggaaaactggtgatccctggtggcatcgacaccagcacccacttccaccagaccttcatgaatgccacgtgcgtggacgacttctaccatgggaccaaggcagcactcgtcggaggcaccaccatgatcatcggccacgtcctgcccgacaaggagacctcccttgtggacgcttatgagaagtgccgaggtctggccgaccccaaggtctgctgtgattacgccctccacgtggggatcacctggtgggcacccaaggtgaaagcagaaatggagacactggtgagggagaagggtgtcaactcgttccagatgttcatgacctacaaggacctgtacatgcttcgagacagtgagctgtaccaagtgttgcacgcttgcaaggacattggggcaatcgcccgcgtccatgctgaaaatggggagcttgtggccgagggtgctaaggaggcactggatttggggatcacaggcccagaaggaatcgagatcagccgtccagaggagctggaagctgaagccactcatcgtgttatcaccattgcaaacaggactcactgtccaatctacctggtcaacgtgtccagtatctcggctggtgacgttatcgcagctgctaagatgcaagggaaggttgtgctggcggagaccaccactgcacatgccacgctgacaggcttacactactaccaccaggactggtcccacgcggctgcctatgtcacggtgcctcccctgagactggacaccaacacctcaacctacctcatgagcctgctggccaatgacactctgaacatcgtggcatcagatcaccggcctttcaccacaaagcagaaagctatgggcaaggaagacttcaccaagatcccacatggagtgagtggcgtgcaggaccgcatgagcgtcatctgggagagaggagtggttggaggaaagatggatgagaaccgttttgtggccgttaccagttccaacgcagctaagcttctgaacctgtatccccgcaagggccgcattattcccggagccgatgctgatgtggtggtgtgggacccagaagccacaaagaccatctcagccagcacgcaggtccagggaggagacttcaacctgtatgagaacatgcgctgccacggcgtgccactggtcaccatcagccgggggcgcgtcgtgtatgagaacggcgtcttcatgtgcgccgagggcaccggcaagttctgtcccctgaggtccttcccagacactgtctacaagaagctggtccagagagagaagactttaaaggttagaggagtggaccgcactccctacctgggggatgtcgctgttgtcgtgcaccctgggaaaaaagagatgggaaccccactcgcagacactcctacccggcccgtcacccggcatgggggcatgagggaccttcacgaatccagcttcagcctctctggctctcagatcgatgaccatgttccaaagcgagcttcagctcggatcctcgctcctcccggaggcaggtcgagtggcatttggtaaaggcattgccaagccccccgagtgaggacgcaccgccgccaccagcccgcaactctccagccgaagctgcaggggcaggagaggctgggctgggtggcacaccacccgaggggggccccgggacccacggagccctccctatgtctgcaaagtgattcactgtgcttcgagccaactctaacaggcactttgagatgtgttcctcctgctgtagtcctttctgccttggcctcggcgggcttttctggggcccaggaagcccacactatgcacagagcccaatgcatagagccctggccagcccttcctctcactcctgcctccgctggctttgggaaagcccagactttagtgccctgccccctggctgactggccagttgcccagagcactttagcagatgtggtttcaaagtaaaggcctcctcccccaccccttaggccccgtggtgacatttcccaagtcagacagatgtcagcttcccagccatgcccaggacgtcctatctcccccaacccacctctggccctgtgtaggggcagggatgggggtggctgggactcctggtgcccctcgccagcttctcctgcgccccgcccacaccctcgggggggtcacaggcccagaagggtagctgggcggggctcgaggctggtgccaggcgcgtgtaaatggttttgttttgcacgtttggtttgcgcagtagtttggtttgacttgtttgtgcatcctgtgaaaaataacggtgcttgtgtcactagcatagaatagcgacaggaatagatgtggtccttaggagacgctgcacttgacaccaaccagacagcacagggcaggggtggtggagggggctgggctcacaggcctctcttttccccgcctgcagtcttctgggctgcgggaggccctggccctttccccttcccctcccctccttgtctagtttcccacattccaaaagggggcctgggatgctagccccagagatgccagcccttcaggaagcaggtgtcctttcccctctctgcccctgatcactcccagcactccccttgccttcccctgtcttcacctgccaccacacacacacacacacacacacacacacacacacgcatggcttcctataacttcttcctgctggacagagactcagcgctcctcctgtgtgactggcaagaggcctcatgcctgctgagagagggtcgacgcggccg

SEQ ID NO: 2 - protein, 564 aa, Homo sapiens
MLANSASVRILIKGGKVVNDDCTHEADVYIENGIIQQVGRELMIPGGAKVIDATGKLVIPGGIDTSTHFHQTFMNATCVDDFYHGTKAALVGGTTMIIGHVLPDKETSLVDAYEKCRGLADPKVCCDYALHVGITWWAPKVKAEMETLVREKGVNSFQMFMTYKDLYMLRDSELYQVLHACKDIGAIARVHAENGELVAEGAKEALDLGITGPEGIEISRPEELEAEATHRVITIANRTHCPIYLVNVSSISAGDVIAAAKMQGKVVLAETTTAHATLTGLHYYHQDWSHAAAYVTVPPLRLDTNTSTYLMSLLANDTLNIVASDHRPFTTKQKAMGKEDFTKIPHGVSGVQDRMSVIWERGVVGGKMDENRFVAVTSSNAAKLLNLYPRKGRIIPGADADVVVWDPEATKTISASTQVQGGDFNLYENMRCHGVPLVTISRGRVVYENGVFMCAEGTGKFCPLRSFPDTVYKKLVQREKTLKVRGVDRTPYLGDVAVVVHPGKKEMGTPLADTPTRPVTRHGGMRDLHESSFSLSGSQIDDHVPKRASARILAPPGGRSSGIW

SEQ ID NO: 3 - DNA, 1829 bp, Homo sapiens
cccaagtccccttcccggcagtttttgccttaaagctgccctcttgaaattaattttttcccaggagagagatgtcttatcaggggaagaaaaatattccacgcatcacgagcgatcgtcttctgatcaaaggaggtaaaattgttaatgatgaccagtcgttctatgcagacatatacatggaagatgggttgatcaagcaaataggagaaaatctgattgtgccaggaggagtgaagaccatcgaggcccactcccggatggtgatccccggaggaattgacgtccacactcgtttccagatgcctgatcagggaatgacgtctgctgatgatttcttccaaggaaccaaggcggccctggctgggggaaccactatgatcattgaccacgttgttcctgagcctgggacaagcctgctcgctgcctttgaccagtggagggaatgggccgacagcaagtcctgctgtgactactctctgcatgtggacatcagcgagtggcataagggcatccaggaggagatggaagcgcttgtgaaggatcacggggtaaattccttcctcgtgtacatggctttcaaagatcgcttccagctaacggattgccagatttatgaagtactgagtgtgatccgggatattggcgccatagcccaagtccacgcagaaaatggcgacatcattgcagaggagcagcagaggatcctggatctgggcatcacgggccccgagggacatgtgctgagccgacctgaggaggtcgaggccgaagccgtgaatcgtgccatcaccatcgccaaccagaccaactgcccgctgtatatcaccaaggtgatgagcaaaagctctgctgaggtcatcgcccaggcacggaagaagggaactgtggtgtatggcgagcccatcactgccagcttgggaacggacggctcccattactggagcaagaactgggccaaggctgctgcctttgtcacctccccacccttgagccctgatccaaccactccagactttctcaactccttgctgtcctgtggagacctccaggtcacgggcagtgcccattgcacgtttaacactgcccagaaggctgtaggaaaggacaacttcaccctgattccggagggcaccaatggcactgaggagcggatgtccgtcatctgggacaaggctgtggtcactgggaagatggatgagaaccagtttgtggctgtgaccagcaccaatgcagccaaagtcttcaacctttacccccggaaaggccgcattgctgtgggatccgatgccgacctggtcatctgggaccccgacagcgttaaaaccatctctgccaagacacacaacagctctctcgagtacaacatctttgaaggcatggagtgccgcggctccccactggtggtcatcagccaggggaagattgtcctggaggacggcaccctgcatgtcaccgaaggctctggacgctacattccccggaagcccttccctgattttgtttacaagcgtatcaaggcaaggagcaggctggctgagctgagaggggttcctcgtggcctgtatgacggacccgtgtgtgaagtgtctgtgacgcccaagacagtcactccagcctcctcggccaagacgtctcctgccaagcagcaggccccacctgtccggaacctgcaccagtctggattcagtttgtctggtgctcagattgatgacaacattccccgccgcaccacccagcgtatcgtggcgccccccggtggccgtgccaacatcaccagcctgggctagagctcctgggctgtgccgtccactggggactggggatgg

SEQ ID NO: 4 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 5 - DNA, 1882 bp, Homo sapiens
ggaggctgcggcgcggccagcgcaccattcactccacctgatctcggggcgctgtgcgctgaggaaggcgcgggcgagccggagcagaagaaggagggagggcgccagccgctgcagccaccaccgccaccatgtcctaccaaggcaagaagaacatcccgcggatcacgagtgaccgtctccttatcaagggaggcagaatcgtcaatgatgatcagtccttttatgctgatatttacatggaagatggcttaataaaacaaattggagacaatgtgattgttcctggaggagtgaagaccattgaagccaatgggaagatggtgatccctggaggcatcgatgtccatactcacttccagatgccatataagggaatgaccacagtagatgacttcttccaagggacaaaggcagccttagcaggtggcaccaccatgatcattgaccatgtggtgcctgagcctgagtccagcctgactgaggcctatgagaaatggagagagtgggctgatgggaagagttgctgtgactatgccctgcatgtggacatcgcccactggaatgacagcgtcaagcaggaagtgcagaacctcatcaaggacaaaggggttaactccttcatggtttatatggcttataaggatttgtatcaagtatctaacacagagctctatgagatcttcacctgcctgggagagctgggggccattgctcaagttcatgctgagaatggggatatcattgcccaggagcaaacccgcatgttggaaatggggataactggcccagaaggccatgtactgagcaggccagaagagctggaagctgaggctgtgttccgtgccatcaccattgccagccaaaccaattgccctctctacgtcacaaaggtcatgagcaagagtgcagctgacctcatctcacaagccaggaaaaaaggaaatgtagtctttggtgagcccatcactgccagcctcggcatagatggaacccattattggagcaagaactgggccaaggcggctgcatttgtgacatccccacccctgagccctgacccaactactccggactacatcaactccttgctggccagcggggatctgcagctatctgggagtgcccactgcaccttcagcactgcccagaaagcaattgggaaggacaacttcacagccattcctgagggcaccaatggtgtggaggagcggatgtctgtcatctgggacaaggctgtggccacagggaaaatggacgaaaaccagttcgtggctgtgacaagcacaaacgctgccaagatcttcaacctgtatccccgcaagggaagaatatctgtgggttctgacagcgacctcgtcatctgggatccagatgctgtgaagatcgtctctgccaagaaccaccagtctgcggcagagtacaacatctttgaagggatggagctgcgcggggctcctctggttgtcatctgccagggcaagatcatgctggaagatggcaacctgcacgtgacccagggggctggccgcttcataccctgcagcccgttctccgactatgtctacaagcgcattaaagcacggaggaagatggcagacctgcatgccgtcccaaggggcatgtacgatgggcctgtgtttgacctgaccaccacccccaaaggtggcacccccgcaggctctgctcggggctctcctactcggccgaacccacctgtgaggaatcttcatcagtcgggatttagcctgtcaggcacccaagtggatgagggggttcgctcagccagcaagcgcatcgtggcgcccccaggcggccgttctaatatcacatctctgagttaagcaagccttcctcaaagagaggggcagaagcaagaaga

SEQ ID NO: 6 - protein, 570 aa, Homo sapiens [sequence omitted]

SEQ ID NO: 7 - DNA, 2842 bp, Homo sapiens
gtgggcatccacgggcgccgagcctccgtccgtgtctctatccctcccgggcctttgtcagcgcgcccgctgggagcggggccgagagcgccggttccagtcagacagccccgcaggtcagcggccgggccgagggcgccagagggggccatgtcgtaccagggcaagaagagcatcccgcacatcacgagtgaccgactcctcatcaaaggtggacggatcatcaacgatgaccaatccctttatgctgacgtctacctggaggatggacttatcaaacaaataggagagaacttaatcgttcctggtggagtgaagaccattgaagccaacgggcggatggttattcccggaggtattgatgtcaacacgtacctgcagaagccctcccaggggatgactgcggctgatgacttcttccaagggaccagggcggcactggtgggcgggaccacgatgatcattgaccatgttgttcctgaacctgggtccagcctactgacctctttcgagaagtggcacgaagcagctgacaccaaatcctgctgtgattactccctccacgtggacatcacaagctggtacgatggcgttcgggaggagctggaggtgctggtgcaggacaaaggcgtcaattccttccaagtctacatggcctataaggatgtctaccaaatgtccgacagccagctctatgaagcctttaccttccttaagggcctgggagctgtgatcttggtccatgcagaaaatggagatttgatagctcaggaacaaaagcggatcctggagatgggcatcacgggtcccgagggccatgccctgagcagacctgaagagctggaggccgaggcggtgttccgggccatcaccattgcgggccggatcaactgccctgtgtacatcaccaaggtcatgagcaagagtgcagccgacatcatcgctctggccaggaagaaagggcccctagtttttggagagcccattgccgccagcctggggaccgatggcacccattactggagcaagaactgggccaaggctgcggcgttcgtgacttcccctcccctgagcccggaccctaccacgcccgactacttgacctccctactggcctgtggggacttgcaggtcacaggcagcggccactgtccctacagcactgcccagaaggcggtgggcaaggacaactttaccctgatccccgagggtgtcaacgggatagaggagcggatgaccgtcgtctgggacaaggcggtggctactggcaaaatggatgagaaccagtttgtcgctgtcaccagcaccaatgcagccaagatctttaacctgtacccaaggaaagggcggattgccgtgggctcggatgccgacgtggtcatctgggaccccgacaagttgaagaccataacagccaaaagtcacaagtcggcggtggagtacaacatcttcgagggtatggagtgccacggctccccactagtggtcatcagccagggcaagatcgtctttgaagacggaaacatcaacgtcaacaagggcatgggccgcttcattccgcggaaggcgttcccggagcacctgtaccagcgcgtcaaaatcaggaataaggtttttggattgcaaggggtttccaggggcatgtatgacggtcctgtgtacgaggtaccagctacacccaaatatgcaactcccgctccttcagccaaatcttcgccttctaaacaccagcccccacccatcagaaacctccaccagtccaacttcagcttatcaggtgcccagatagatgacaacaatcccaggcgcaccggccaccgcatcgtggcgccccctggtggccgctccaacatcaccagcctcggttgaacgtggatgcgcggaggagctagcctgaaggattctgggaatcatgtccatcccttttcctgtcagtgtttttgaaacccacagttttagttggtgctgatggagggagggggaagtcgaaggatgctctttcccttttctgtttaggaagaagtggtactagtgtggtgtgtttgcttggaaattccttgccccacagttgtgttcatgctgaatccacctcggagcatggtgttttcattcccccttcctagtgaaccacaggttttagcattgtcttgttctgtcccttccacttctaactccactggctccatgattctctgagtggtggttcctttgcaccctgtagatgttctaggatagttgatgcatgttactaaattacgtatgcaagtctgtgagtgcgtctgaggggacatcgccaaggactgactgagacacgatgccgagacctcaagccctgaggggcagtcccaaaacccttacagtgaagatgtttactcattgcccccacctctggtccacactagaaagaagctcgccccacctccacctgtgagatccgtgaattctcggaatggcaggggaagccttgcactaggttgcagagaagcatcctccacatcctgtgtcagaaaccctggtctccgtggcacttgtaactcaccgtgctgtcttctggtctgtgtgtgttcttcaagccagctctaggcttcaggccgagccaggttcacactcagaaagatgtctccccatccccattcggggctgacgatggggggctgatggctgcccctgcgtggcctgagtcctggtccctctgaggcagttgacggggcagtcagatttttaaagttttgtacaaagttttcctttgtaatcactcccatttttacttaacaaccaacttgttgtggctcttatttctgaattcaaagcttgtgaaaaaataaaagaaaatgaactgccc

SEQ ID NO: 8 - protein, 572 aa, Homo sapiens
MSYQGKKSIPHITSDRLLIKGGRIINDDQSLYADVYLEDGLIKQIGENLIVPGGVKTIEANGRMVIPGGIDVNTYLQKPSQGMTAADDFFQGTRAALVGGTTMIIDHVVPEPGSSLLTSFEKWHEAADTKSCCDYSLHVDITSWYDGVREELEVLVQDKGVNSFQVYMAYKDVYQMSDSQLYEAFTFLKGLGAVILVHAENGDLIAQEQKRILEMGITGPEGHALSRPEELEAEAVFRAITIAGRINCPVYITKVMSKSAADIIALARKKGPLVFGEPIAASLGTDGTHYWSKNWAKAAAFVTSPPLSPDPTTPDYLTSLLACGDLQVTGSGHCPYSTAQKAVGKDNFTLIPEGVNGIEERMTVVWDKAVATGKMDENQFVAVTSTNAAKIFNLYPRKGRIAVGSDADVVIWDPDKLKTITAKSHKSAVEYNIFEGMECHGSPLVVISQGKIVFEDGNINVNKGMGRFIPRKAFPEHLYQRVKIRNKVFGLQGVSRGMYDGPVYEVPATPKYATPAPSAKSSPSKHQPPPIRNLHQSNFSLSGAQIDDNNPRRTGHRIVAPPGGRSNITSLG

SEQ ID NO: 9 - DNA, 1690 bp, Homo sapiens
gccgcccctaccagagacccccaggagcaggatgtccttccagggcaagaaaagcatcccccggatcacgagtgaccgccttctgatcagaggtgggaggatcgtgaatgacgaccagtccttttacgctgatgtgcacgtggaagatggcttgataaaacaaatcggagaaaacctcatcgtccctgggggcatcaagaccattgacgcccacggcctgatggtccttcctggtggcgttgacgtccacacaaggctgcagatgcctgtcctgggcatgacaccggctgacgacttctgtcagggcaccaaggcagcgctagcaggaggaaccaccatgatcttggaccacgtcttccccgacacgggtgtgagcctgctggcggcctacgagcagtggcgggagcgggcggacagcgcggcctgctgcgactactccctgcacgtggacatcacccgatggcatgagagcatcaaggaggagctggaggccctggtcaaggagaagggtgtgaactccttcctggtcttcatggcatacaaggaccggtgccagtgcagcgacagccagatgtacgagatcttcagcatcatccgggacctgggggccttggcccaggtgcacgctgagaacggggacatcgtggaggaggagcagaagcggttgctggagctcggcatcactggccccgagggccacgtgctcagccaccccgaggaggtggaggctgaggcggtgtaccgagctgtcaccatcgccaagcaggcaaactgcccgctgtacgtcaccaaggtgatgagcaagggggcggccgacgccatcgctcaggccaagcgcagaggggtggtcgtgtttggggagcccatcaccgccagcctgggcaccgacggttcacactactggagcaagaactgggccaaggctgcagccttcgtcacatcaccccctgtcaacccagaccccaccacggcagaccacctcacctgcttgctgtccagcggggacctccaggtgacaggcagcgcccactgcaccttcaccactgcccagaaggctgtgggcaaggacaacttcgcgctgatccccgagggcaccaacggcattgaggagcgcatgtcgatggtctgggagaaatgtgtggcctctgggaagatggacgagaatgagttcgtcgcggtgaccagtacaaatgctgccaaaatcttcaatttttacccaaggaaggggcgagtggctgtgggctctgacgctgacctggtcatatggaaccccaaggccaccaagatcatctctgccaagacccacaatctgaacgtggagtacaacatcttcgagggagtggagtgccggggagcgcctgccgtggtcataagtcagggccgagtggcgctggaggacgggaagatgtttgtcaccccgggggcgggccgcttcgtccctcggaaaacattcccggactttgtctacaagaggatcaaagctcgcaacaggctggcggagatccacggtgtgccccgtgggctgtatgacgggcccgtccacgaggtgatggtgcctgccaagccagggagtggcgctccggcccgcgcgtcctgcccaggcaagatctccgtgcctcctgtgcgcaacctacatcagtcggggttcagcctatctgggtctcaggctgatgaccacatcgcccgacgc

SEQ ID NO: 10 - protein, 572 aa, Homo sapiens
MSFQGKKSIPRITSDRLLIRGGRIVNDDQSFYADVHVEDGLIKQIGENLIVPGGIHTIDAHGLMVLPGGVDVHTRLQMPVLGMTPADDFCQGTKAALAGGTTMILDHVFPDTGVSLLAAYEQWRERADSAACCDYSLHVDITRWHESIKEELEALVKEKGVNSFLVFMAYKDRCQCSDSQMYEIFSIIRDLGALAQVHAENGDIVEEEQKRLLELGITGPEGHVLSHPEEVEAEAVYRAVTIAKQANCPLYVTKVMSKGAADAIAQAKRRGVVVFGEPITASLGTDGSHYWSKNWAKAAAFVTSPPVNPDPTTADHLTCLLSSGDLQVTGSAHCTFTTAQKAVGKDNFALIPEGTNGIEERMSMVWEKCVASGKMDENEFVAVTSTNAAKIFNFYPRKGRVAVGSDADLVIWNPKATKIISAKTHNLNVEYNIFEGVECRGAPAVVISQGRVALEDGKMFVTPGAGRFVPRKTFPDFVYKRIKARNRLAEIHGVPRGLYDGPVHEVMVPAKPGSGAPARASCPGKISVPPVRNLHQSGFSLSGSQADDHIARRTAQKIMAPPGGRSNITSLS

SEQ ID NO: 11 - protein, 20 aa, Artificial Sequence
  Description of Artificial Sequence immunogenic peptide
KEMGTPLADTPTRPVTRHGG

SEQ ID NO: 12 - protein, 12 aa, Artificial Sequence
  Description of Artificial Sequence immunogenic peptide
LEDGTLHVTEGS

SEQ ID NO: 13 - protein, 16 aa, Artificial Sequence
  Description of Artificial Sequence immunogenic peptide
ITGPEGHVLSRPEEVE

SEQ ID NO: 14 - protein, 15 aa, Artificial Sequence
  Description of Artificial Sequence immunogenic peptide
LTSFEKWHEAADTKS

SEQ ID NO: 15 - protein, 13 aa, Artificial Sequence
  Description of Artificial Sequence immunogenic peptide
EHDSHAQLRWRVL

SEQ ID NO: 16 - DNA, 25 bp, Artificial Sequence
  Description of Artificial Sequence primer
atagacacgatgccaagaccttacc

SEQ ID NO: 17 - DNA, 22 bp, Artificial Sequence
  Description of Artificial Sequence primer
attaccgcaccatcctcaaggc

SEQ ID NO: 18 - DNA, 24 bp, Artificial Sequence
  Description of Artificial Sequence primer
atcacccatcccttactcttctgg

SEQ ID NO: 19 - DNA, 25 bp, Artificial Sequence
  Description of Artificial Sequence primer
cagaagaaaaagccagaacagaccg

SEQ ID NO: 20 - DNA, 25 bp, Artificial Sequence
  Description of Artificial Sequence primer
cccctccccataaactctcttttgg

SEQ ID NO: 21 - DNA, 20 bp, Artificial Sequence
  Description of Artificial Sequence primer
ctggaaagttcacaggctgg

SEQ ID NO: 22 - DNA, 25 bp, Artificial Sequence
  Description of Artificial Sequence primer
cctaccagggcaagaagaacattcc

SEQ ID NO: 23 - DNA, 22 bp, Artificial Sequence
  Description of Artificial Sequence primer
ccgcaatggtcttcacacctcc

SEQ ID NO: 24 - DNA, 21 bp, Artificial Sequence
  Description of Artificial Sequence primer
ctgtggatgtggacatgaagc

SEQ ID NO: 25 - DNA, 22 bp, Artificial Sequence
  Description of Artificial Sequence primer
agcaataaacaggtggaaggtc

SEQ ID NO: 26 - DNA, 17 bp, Artificial Sequence
  Description of Artificial Sequence primer
agagagattcgcactca

SEQ ID NO: 27 - DNA, 19 bp, Artificial Sequence
  Description of Artificial Sequence primer
agtgcctcctggtaactgg

SEQ ID NO: 28 - DNA, 20 bp, Artificial Sequence
  Description of Artificial Sequence primer
gaagagtggttcaagagccg

SEQ ID NO: 29 - DNA, 25 bp, Artificial Sequence
  Description of Artificial Sequence primer
tgccatcttgacattgaggaggtcc

SEQ ID NO: 30 - DNA, 30 bp, Artificial Sequence
  Description of Artificial Sequence antisense
cagacatgtgataccagaaggtcatgcagt